(19) United States
(12) Patent Application Publication
(10) Pub. No.: US 2020/0253723 A1
(43) Pub. Date: Aug. 13, 2020

(54) PUPILLARY ACCOMMODATING INTRAOCULAR LENS

(71) Applicants: Douglas Michael Ackermann, Reno, NV (US); Anand Doraiswamy, San Francisco, CA (US)

(72) Inventors: Douglas Michael Ackermann, Reno, NV (US); Anand Doraiswamy, San Francisco, CA (US)

(21) Appl. No.: 16/635,530

(22) PCT Filed: Jul. 27, 2018
(86) PCT No.: PCT/US2018/044236

§ 371 Date: Jan. 30, 2020

Related U.S. Application Data
(60) Provisional application No. 62/539,498, filed on Jul. 31, 2017.
(60) Provisional application No. 62/594,491, filed on Dec. 4, 2017.
(60) Provisional application No. 62/666,584, filed on May 3, 2018.

(51) Int. Cl. A61F 2/16

(52) U.S. Cl. CPC A61F 2/1635; A61F 2002/1681; A61F 2/1608

(57) ABSTRACT

Systems and methods can advantageously leverage the pupillary accommodation reflex to provide improved quality of vision throughout the range of focus distances. As such, some embodiments include an accommodating pupillary lens (APL) that sits at least partially within the pupil and/or is partially or fully attached, adhered, or otherwise held in place with respect to the iris, and/or is anchored in the sulcus and/or elsewhere in the eye. The optical power of the lens can be configured to change with changes in pupil diameter.

This application claims the benefit under 35 U.S.C. § 119(e) as a nonprovisional application of each of the following provisional applications: U.S. Prov. App. No. 62/539,498, filed on Jul. 31, 2017; U.S. Prov. App. No. 62/594,491 filed on Dec. 4, 2017; and. U.S. Prov. App. No. 62/666,584 filed on May 3, 2018, each of which are incorporated by reference in their entireties.

### BACKGROUND

Presbyopia is the loss of nearsighted vision caused by loss of elasticity of the lens of the eye, usually occurring in middle age (e.g., around 40-50 years of age) and typically continues to worsen into the 60s. It has been estimated that over 100 million people in the US have presbyopia and over 1.3 billion people worldwide, and the number is expected to increase dramatically as the population ages. Presbyopia can be corrected by glasses, contact lenses, or surgical lens replacement, e.g., intraocular lens implantation, which is typically performed for cataracts, among other indications. There are various types of intraocular lenses (IOLs) present.

Some conventional IOLs are monofocal lenses, designed to provide clear vision at a single focal point (e.g., typically far vision). With conventional IOLs, typically a user is still dependent on vision correction at other focal points. For example, patients with conventional monofocal IOLs can be required to wear eyeglasses or contact lenses in order to see clearly for near vision tasks, such as reading.

Multifocal IOLs can improve vision at both near and far distances, but still can have several disadvantages, including blurriness at some distances, such as very near distances, decreased low light vision, glare, halos, and starbursts. Also, far distance vision may not be as sharp as that of monofocal IOLs. Tonic IOLs have different powers in different meridians of the lens to correct the asymmetric power of the eye that is characteristic of astigmatism.

Accommodating IOLs are configured to move and flex with ocular muscle contraction, to accommodate similar to the eye's natural lens, allowing for focusing throughout the range of vision. However, accommodating IOLs can have complications such as Z syndrome and anterior or posterior dislocations. Patients can also sometimes experience glare and halos from a smaller optic zone and irregular astigmatism related to optic flexure. Furthermore, predicting effective lens position due to the haptic configuration can be difficult the lens optic may be slightly more posterior or anterior than anticipated due to anatomical issues such as capsular size. Moreover, undesired lens migration and inadequate haptic movement or flexing causing vaulting can also be potential issues. Furthermore, the ciliary muscle may not be strong enough to power the lens to provide proper focus throughout the full range of vision distances. As such, improved accommodating IOLs and methods of implantation and use are needed.

### SUMMARY

Some embodiments of the invention advantageously leverage the pupillary accommodation reflex to provide improved quality of vision throughout the range of focus distances. As such, some embodiments of the invention include an accommodating pupillary intraocular lens (APL or APIOL) that sits at least partially within the pupil and/or is partially or fully attached, adhered, or otherwise held in place with respect to the iris, and/or is anchored in the sulcus and/or elsewhere in the eye. The optical power of the lens can be configured to change with changes in pupil diameter. In some embodiments, the optical power of the lens can include a sufficient range of accommodation of up to at least about 1, 2, 3, 4, 5, 6, 7, 8, 9, 10, 11, 12, 13, 14, 15 diopters, or ranges including any two of the aforementioned values. The forces of the ciliary body, iris movements, and/or pupillary changes can be translated to change the optical power of the lens. In some embodiments, the system can be configured to diminish pupillary changes resulting from the light reflex. In some embodiments, pupil changes can result in power changes driven by axial displacement of the lens optics, change in vertex distance, and/or curvature changes, resulting in accommodation. The accommodating pupillary lens can be configured to fit a specific patient's range in pupil size (e.g., between scotopic and photophic conditions) in some embodiments.

In some embodiments, the lens could include one, two, or more fixation elements. The fixation elements can promote fixation of the lens to the iris. In some embodiments, the iris fixation dements can include a pinch mechanism. In some embodiments, the iris fixation elements may include a lip and/or groove for stabilizing and/or fixing the lens at the pupillary margin. The fixation elements can also promote fixation of the lens within the sulcus. The fixation elements can include one, two, or more haptics. The fixation elements can also include sub-elements to anchor the lens to the iris, for example, grooves, teeth, ridges, or a saw-tooth pattern, for example. In some embodiments, two or more of the same or different fixation elements can be used in combination (for example, sulcus haptics and grooves to hold the pupillary margin of the iris).

In some embodiments, the lens can include grooves/holes/perforations to allow fluid transfer between the anterior and posterior chamber. The lens material may have part of the optic zone partially or completed blocked off to visible light to reduce photophobia while improving accommodation. The lens material may have part of the optic zone partially or completed blocked off by a photochromic or light-sensitive material/chromophore. The lens design can, in some embodiments, utilize and leverages the pupillary movements (accommodative reflex) to result in desired change in power of the lens and system. The haptic(s) of the lens can additionally anchor strategically away from the iris into the sulcus to allow stability and use of ciliary forces upon accommodative demand. In some embodiments, a gel-filled multi-material lens system utilizes the forces from the pupillary constriction and ciliary body to result in desired optic power change. The lens can be used as a piggy-back lens to provide accommodation on top of the existing in-the-bag lens. The APIOL can also be used as a stand-alone accommodating lens without the need for a regular in-the-bag lens.

Also disclosed herein are methods of treating presbyopia by positioning an accommodating pupillary intraocular lens within the eye of a subject. The haptics can rest on, contact, attach (such as being sutured or anchored to), adhere to, or otherwise be connected partially or entirely on the iris and/or ciliary body, the sulcus, the angle, the capsular bag, or not at all on any number of these locations in some embodiments. The lens could be used alone (e.g., be entirely extracapsular), or in combination with an intracapsular IOL in some cases. The accommodating pupillary intraocular lens can, in some embodiments, be implanted with a placement tool configured to manipulate haptics to rest on the iris. The accommodating pupillary lens could serve, for example, as a pseudophakic lens, a piggy-back lens (in combination with a pseudophakic lens), or a phakic lens. In some embodiments, the lens can be placed only in one eye (e.g., monocularly) to provide desired balance between 3D binocular vision and distance/near accommodation. In some embodiments, placement of a lens in only one eye (e.g., a non-dominant eye) can reduce side effects such as lens power changes with iris movements not associated with focus depth. In other embodiments, lenses can be placed binocularly. The lens can treat one or more of regular astigmatism, irregular astigmatism, myopia, hyperopia, and/or other orders of monochromatic or chromatic aberration, including but not limited to spherical aberration, defocus, field curvature, image distortion, axial or lateral chromatic aberration. In sonic embodiments, the lens can include a hole in the middle of the optic zone, and/or perforations or gaps in the periphery of the lens to provide adequate aqueous flow and transfer. As such, some embodiments can allow for a mechanism for fluid transfer of a lens sitting in the pupillary plane interfacing with the iris. In some embodiments, the lens material can have a responsive refractive index, e.g., change in refractive index reactive to forces from the iris and/or ciliary body. The lens material can have a variable refractive index across the optic zone. The lens material can have part of the optic zone partially or completely blocked off from visible light or a subset of visible light wavelengths to reduce photophobia while improving accommodation. The lens material can also have part of the optic zone be partially or completely blocked off by a photochromic or light-sensitive material/chromophore.

In some embodiments, disclosed is a method of surgically implanting an accommodating pupillary intraocular lens. A measuring device can be used to measure the compressive and/or tensile force of the iris prior to fitting the lens. The lens can be implanted using a small or micro-incision in the limbus region or other parts of the cornea. The lens can be implanted via a placement tool allowing manipulation of haptics (e.g., lateral ends of the haptics) to rest on the iris and/or within the sulcus in some cases. The lens could be implanted alone, or in combination with an intracapsular IOL in some embodiments.

The accommodating lens can be customized for a specific patient, including iris factors (e.g., color, pigmentation, thickness, roughness, uniformity, circularity, tissue, age, and other factors unique to the patient).

### BRIEF DESCRIPTION OF THE DRAWINGS

FIG. 1A illustrates a graph showing the relationship between the direct light reflex amplitude vs. stimulus intensity in normal subjects. FIG. 1B is a graph illustrating pupil diameter with respect to light intensity.

The size of the pupil (in photopic, mesopic, and scotopic conditions) generally decreases with age, as demonstrated in the attached tables and graphs of FIG. 2.

FIGS. 3A-3C illustrate lens embodiments that can be configured to adjust optical power of a lens-eye system with a change in pupil diameter.

FIG. 3D illustrates a graphic showing that pupil diameter (solid line) increases from near focus to far focus, while optical power (dashed line) of the lens decreases respectively.

FIG. 4A illustrates a side view of another embodiment of an accommodating pupillary intraocular lens with a plurality of lenses, FIG. 4B illustrates the accommodating pupillary intraocular lens of FIG. 4A illustrating transformation of the lens between a far focus/gaze configuration (on the left) and a near focus/gaze configuration (on the right).

FIG. 5A illustrates a side view of another embodiment of an accommodating pupillary intraocular lens including haptics that can take the form of crossing leg members. FIG. 5B illustrates the lens transforming to a decreased radius of curvature configuration as forces from the iris IR result in the leg members of the haptics exert compressing the optic.

FIGS. 6A-6F are top views of different embodiments of accommodating intraocular lenses.

FIGS. 7A-7F illustrates further embodiments of accommodating intraocular lenses.

FIGS. 8A-8B illustrate graphs relating to pupillary response relative sensitivity to light of various wavelengths.

FIG. 8C illustrates non-limiting example ranges of lens system filter characteristics.

FIG. 8D is another diagram illustrating the relative sensitivity of the pupillary response with and without a selective chromatic IOL filter, according to some embodiments.

FIG. SE is a schematic graph illustrating that a change in optical power of the lens can be dampened at relatively large pupil diameters to allow for selective movement of the lens to the near-accommodative reflex.

FIG. 8F illustrates a top view of an accommodating lens system with only one optic (or a plurality of optics in other embodiments) and at least a first haptic and a second haptic, the first and second haptics being not identical in one or more features with respect to each other.

FIG. 8G is a side view of the embodiment of FIG. 8F, with only a single haptic of each type illustrated for clarity.

FIGS. 9A-9B illustrate an embodiment of an iris-fixated accommodating pupillary lens, according to some embodiments of the invention.

FIGS. 10A-10B illustrate views of another embodiment of an iris-fixated accommodating pupillary lens.

FIGS. 11A-11I illustrate views of embodiments of an accommodating pupillary lens configured to be placed in the ciliary sulcus.

FIGS. 12A-12K illustrate various embodiments of accommodating pupillary lens systems.

FIGS. 13A-13I illustrate various embodiments of accommodating pupillary lens systems with external covering layers and media contained within.

FIGS. 14A-14D illustrate another embodiment of an accommodating pupillary lens with a dual vault.

FIGS. 15A-15D illustrate another embodiment of an accommodating pupillary lens with one or a plurality of optics and spaced-apart haptics extending radially outward from the optic.

FIGS. 16A-16I illustrate further schematic illustrations of embodiments of an accommodating pupillary lens system including one or more meniscus portions.

FIGS. 17A-17C illustrate schematic illustrations of embodiments of an accommodating pupillary lens system with accordion-like hinges.

### DETAILED DESCRIPTION

Several factors that influence (e.g., increase and/or decrease) pupil diameter have been identified. One is the Pupillary Light Reflex (PLR). The pupillary light reflex (PLR) or photopupillary reflex controls the diameter of the pupil, in response to the intensity (luminance) of light mediated by the retinal photoreceptors, thereby assisting in adaptation to various levels of lightness/darkness. Another is the Accommodation Reflex (AR) or Near Reflex (NR): AR is a synkinesis, rather than a true reflex. Activated in response to focusing on near object, AR involves a coordinated change in vergence, lens shape, and pupil size. Yet another is pupillary hippus (pupillary athetosis), which is pupillary unrest (e.g., spasmodic rhythmic pupillary movements) due to oscillations in the balance between parasympathetic nervous system (PNS) and the sympathetic nervous system (SNS). Still another is psychosensory input: dilation of pupil in response to sensory and psychic stimuli. Sympathetic discharge to the dilator pupillae and/or inhibition of the parasympathetic discharge to the sphincter pupillae can lead to pupil dilation. A further factor is cognitive load: the change in pupil size in response to mental activity and memory load.

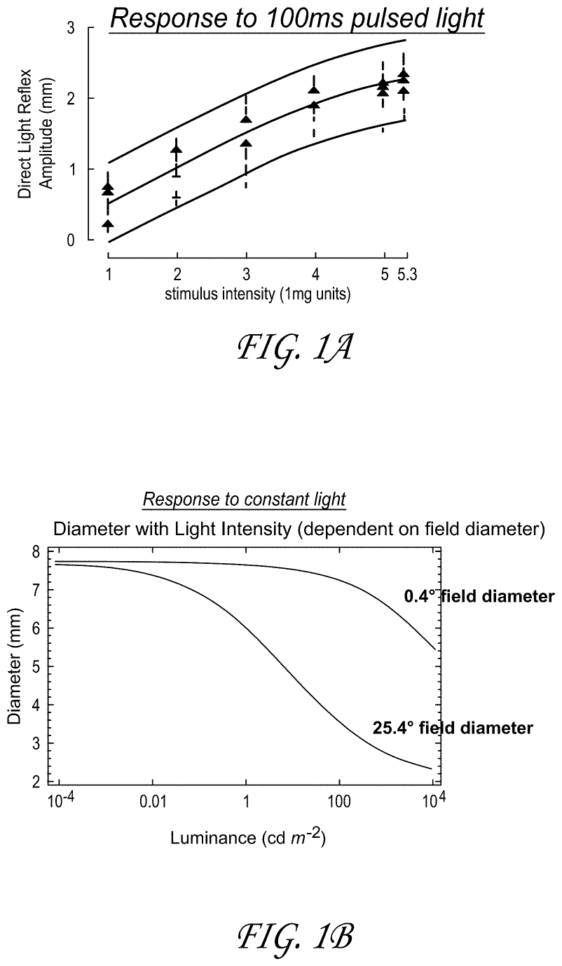

Of the foregoing, the Pupillary Light reflex (PLR) and Accommodation Reflex (AR) or Near Reflex (NR) are the primary drivers of pupil diameter. FIG. lA illustrates a graph showing the relationship between the direct light reflex amplitude vs. stimulus intensity in normal subjects. FIG. 1B is a graph illustrating pupil diameter with respect to light intensity. The pupil diameter can be to a first approximation dependent on the product of luminance and adapting field area, or corneal flux density. When light is limited to about <20 cd/m², the pupil diameter can remain above 4 mm even at the widest field (as shown via the arrow), leaving about 2 more millimeters for focus-induced changes. As such, a light-filtering lens can be configured to limit the optical power on the retina.

Other drivers are generally less significant in magnitude, and generally dilatory in nature (meaning shift to far-field focus and therefore less of an impact on design), particularly when the pupil is under active constriction. For example, hippus typically results in less than about <0.5 mm changes; both dilatory and constrictive to pupil. Psychosensory factors can be in response to a wide array of stimuli and are typically dilatory. Cognitive load is typically less than about <0.5 mm and is typically dilatory to the pupil.

The accommodation reflex, or near reflex, assists in focusing on objects close to the eyes, and is a neural response to unfocused foveal image & binocular disparity. Three motor processes occur with near focus: (1) ciliary muscle contraction to relax tension on zonule fibers & allow the lens to increase curvature; (2) contraction of the medial rectus to adjust gaze more nasally; and (3) constriction of the iris sphincter. Efferent fibers originate in Edinger-Westphal nucleus and travel via the oculomotor nerve, and may not synapse in the ciliary ganglion in some cases.

The size of the pupil (in photopic, mesopic, and scotopic conditions) generally decreases with age, as demonstrated in the attached tables and graphs of FIG. 2. While lens accommodation diminishes with age, the pupillary accommodation reflex generally remains (although the range of pupil size changes—as the pupil can shrink with age). The accommodation reflex is most sensitive to blue and green light, with sharp decrease in sensitivity beyond green (e.g., above about 570 nm wavelengths).

The pupil size diminishes with an increase in luminance. This effect (delta) is diminished with age, although still functional. One way to achieve an accommodation reflex alone is to filter the wavelengths responsible for light reflex. However, at photopic conditions, since the pupil is small, light is parallel and accommodative refractive demand for near sight is riot high. At scotopic conditions, since the pupil is largest, most of accommodation is achieved through the near reflex.

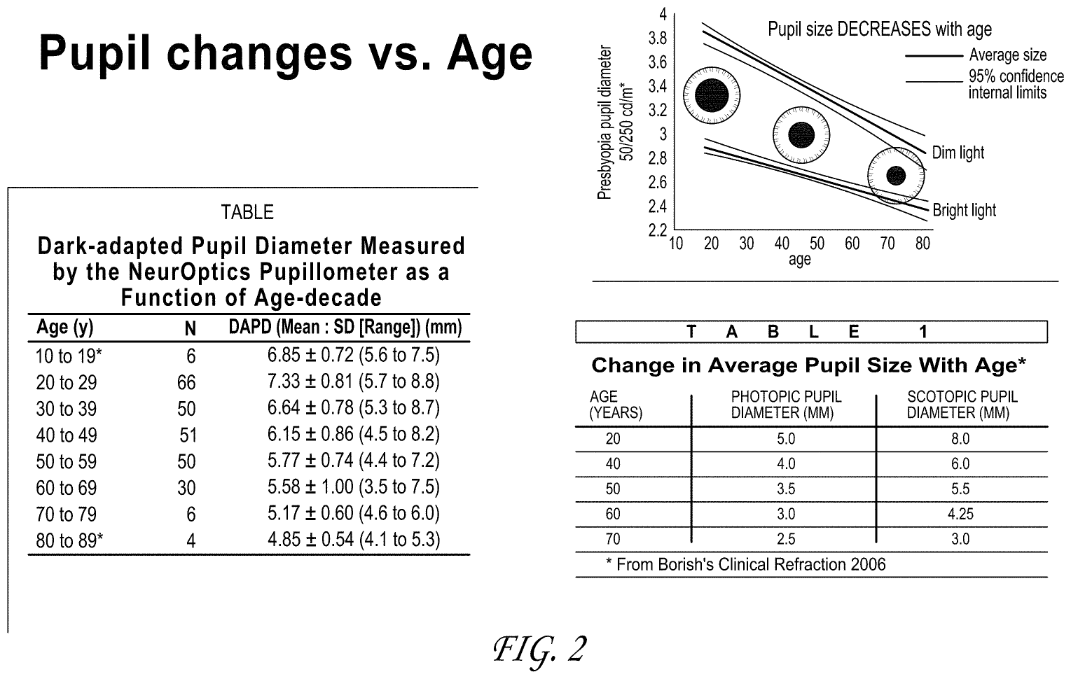

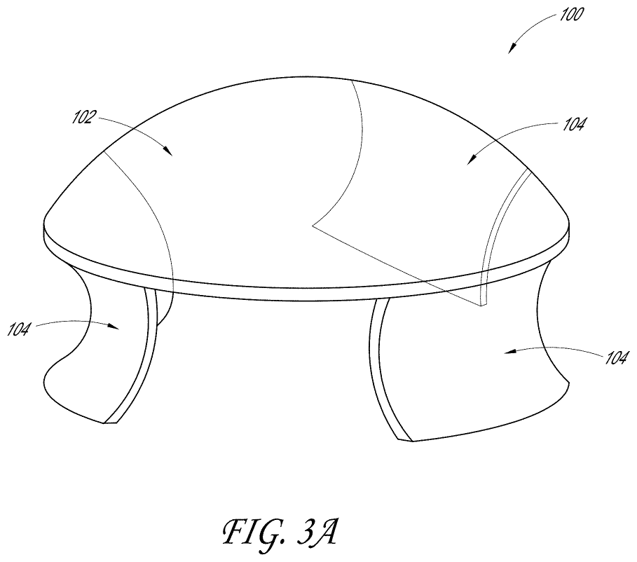

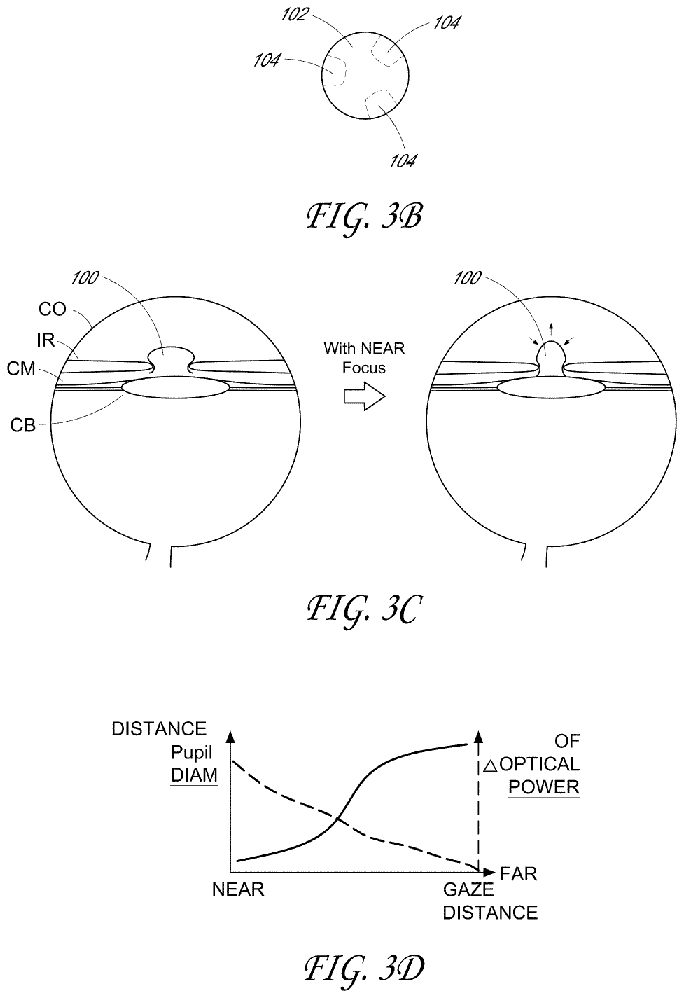

Disclosed herein are lens embodiments that can be configured to adjust optical power of a lens-eye system with a change in pupil diameter. FIG. 3A is a side view of an embodiment of a pupillary accommodating intraocular lens 100. The lens 100 can include an optic 102 and a plurality of haptic portions 104 (such as 3 haptic portions 104 as shown) operably attached to the optic 102. In some embodiments, the haptic portions 104 extend posteriorly with respect to the optic 102 and can be curved, such as concave radially outwardly as shown in some cases. The haptic portions can be configured to attach to the peripheral edge of the pupil, e.g., on a portion of the iris. FIG. 3B is a top view of the lens 100, with optic 102 and haptic portions 104 as previously described. In some embodiments, the lens 100 could include 1, 2, 3, 4, or more pupillary haptics 104. FIG. 3C illustrates an accommodating pupillary intraocular lens 100 implanted within the eye proximate the iris IR, and also illustrating the cornea CO, ciliary muscle CM, and capsular bag CB, illustrating transformation of the lens 100 between a far focus/gaze configuration (on the left) and a near focus/gaze configuration (on the right). For far focus/gaze, the iris IR is relaxed, resulting in an increased pupil diameter, less force on the lens 100, resulting in a larger radius of curvature of the lens 100. As illustrated by arrows (right frame), constriction of the iris IR decreases the pupil diameter and exerts lateral compression forces on the lens 100, dynamically decreasing the radius of curvature of the lens. FIG. 3D illustrates a graphic showing that pupil diameter (solid line) increases from near focus to far focus, while optical power (dashed line) of the lens 100 decreases respectively.

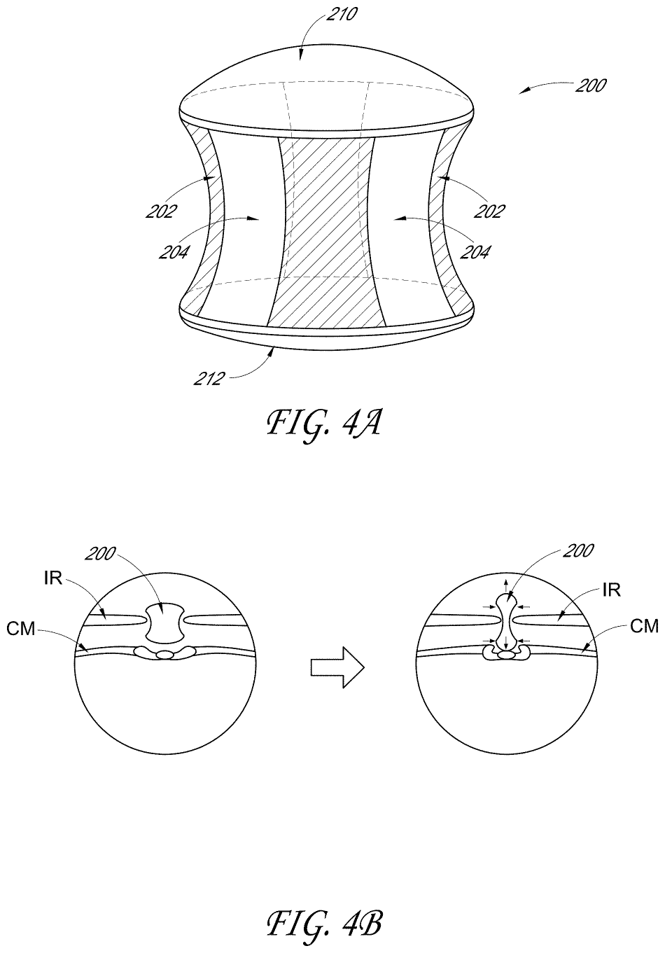

FIG. 4A illustrates a side view of another embodiment of an accommodating pupillary intraocular lens 200 with a plurality of lenses, including an anterior optic 210 and a posterior optic 212, interconnected by one or a plurality (e.g., 2, 3, 4, or more) haptics 202, such as 4 haptics as shown. One or both of the plurality of optics 210, 212, can be configured to change their radius of curvature with pupillary dilation and constriction as previously described. In some embodiments, apertures or gaps 204 can be present between the haptics 202 to allow for aqueous humor to flow through the lens. FIG. 4B illustrates the accommodating pupillary intraocular lens 200 of FIG. 4A illustrating transformation of the lens 200 between a far focus/gaze configuration (on the left) and a near focus/gaze configuration (on the right) with iris IR and/or ciliary muscle CM contraction as previously described. In FIG. 413 the natural lens is illustrated removed, although a natural or intracapsular lens can also be present in other embodiments. In some embodiments, the lens could be a solid plug construction (e.g., a single continuous body) with or without cutout sections for aqueous flow around the plug lens. In some embodiments, one or more of the haptics could be used to stabilize the accommodating pupillary intraocular lens and/or maintain centricity, while one or more of the other haptics could be used to modulate pupil constriction into axial or other changes of the lens.

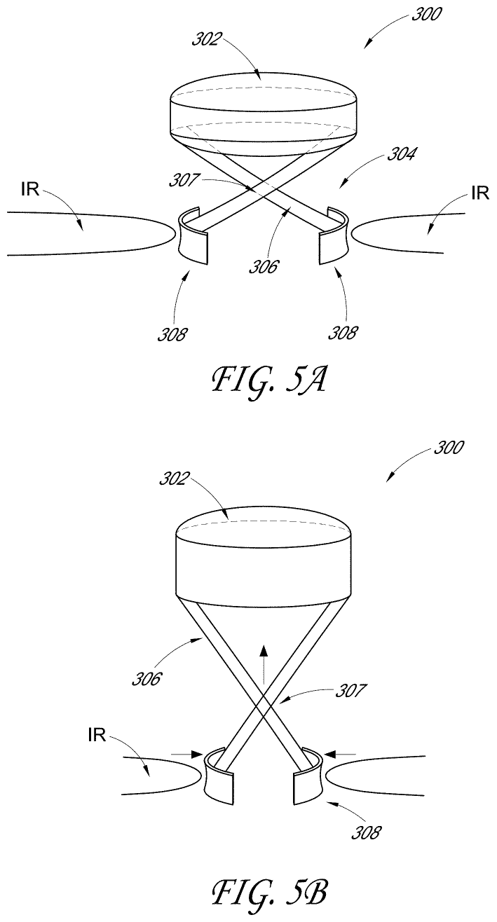

FIG. 5A illustrates a side view of another embodiment of an accommodating pupillary intraocular lens 300 with an optic 302 that can be configured to be positioned axially anterior to the iris, and a plurality (e.g., 2, 3, 4, or more) haptics 304 that can take the form of crossing leg members 306 as shown, as well as ends 308 that can be configured to rest against or attach to the iris IR at the periphery of the pupil. The leg members 306 can intersect at a hinge or pivot point 307 in some cases. The ends 308 can include convex radially outwardly as shown, or flat, concave, or other geometries. In some embodiments, the haptics 304 can be positioned outside of the optical axis. FIG. 5A illustrates the lens 300 in a far gaze configuration with a relatively large pupil diameter. FIG. 5B illustrates the lens 300 transforming to a decreased radius of curvature configuration as forces from the iris IR result in the leg members 306 of the haptics 304 exert compressing a portion of, such as the peripheral edge of the optic 302. As shown, the iris IR does not directly contact the optic 302 in some embodiments, but rather forces from iris constriction indirectly translate to the optic 302 via the leg members 306 of the haptic.

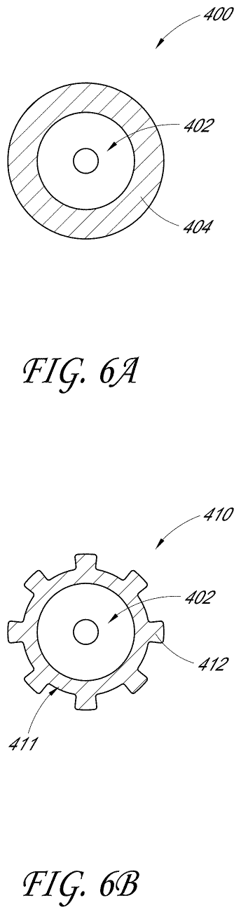

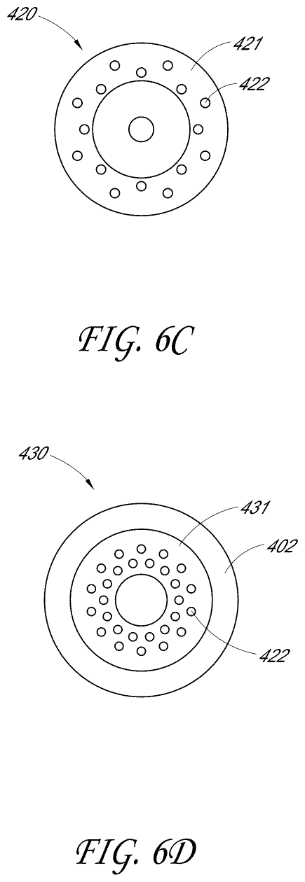

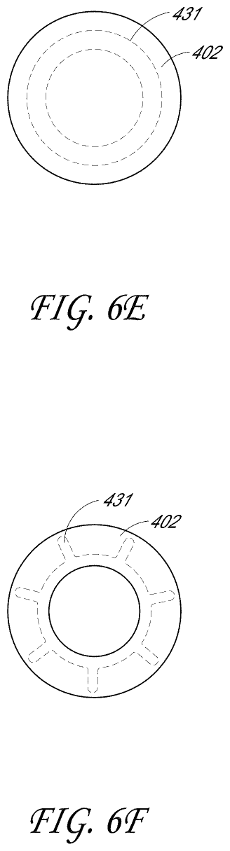

FIGS. 6A-6F are top views of different embodiments of accommodating intraocular lenses. FIG. 6A illustrates a lens 400 with an optic 402 that is partially or completely circumferentially surrounded by at least one haptic 404 such that the diameter of the haptic 404 is greater than the diameter of the optic 402. FIG. 6B illustrates a lens 410 with an optic 402, a haptic circumferential base portion 411 with spaced-apart haptic projections 412 at least partially or completely circumferentially surrounding the optic 402. The haptics can include projections 412 extending radially outwardly from the optic 402. The diameter of the haptic can be greater than the diameter of the optic 402. FIG. 6C illustrates an embodiment of a lens 420 similar to that shown in FIG. 6A, with apertures or perforations 422 in the haptic 421 configured to allow circumferential fluid (e.g., aqueous humor) flow through the haptic 421. In some embodiments, the surface area of the perforations can be about 1%, 2%, 3%, 4%, 5%, 6%, 7%, 8%, 9%, 10%, 15%, 20%, 30%, 40%, 50%, 75%, or more of the surface area of the haptic, or ranges incorporating any two of the aforementioned values. FIG. 61) illustrates another embodiment of a lens 430 where a portion of the haptic 431 is circumferentially surrounded by the optic 402. The haptic can include apertures or perforations 422 as previously described, or lack any apertures or perforations as illustrated in FIG. 6E. FIG. 6F illustrates an embodiment similar to FIG. 613, except that the optic 402 is radially outward of the haptics 431, in contrast to the optic being radially inward of the haptics as illustrated in FIG. 6B. The haptic diameter of the FIGS. 6D-6F embodiments can be less than the optic diameter, in contrast to the embodiments of FIGS. 6A-6C where the haptic diameter is greater than the optic diameter. In other embodiments, the haptic maximal or average diameter can be substantially equal to the diameter of the optic. in some embodiments, the diameter of the optic can be about, at least about, or no more than about 5%, 10%, 15%, 20%, 25%, 30%, 35%, 40%, 45%, 50%, 75%, 100%, or more greater or less than the diameter of the haptic, or ranges incorporating any two of the aforementioned values.

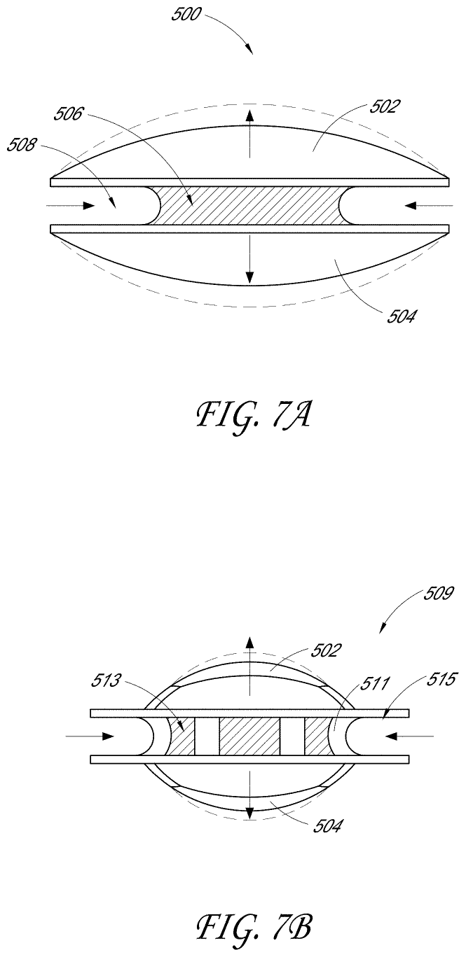

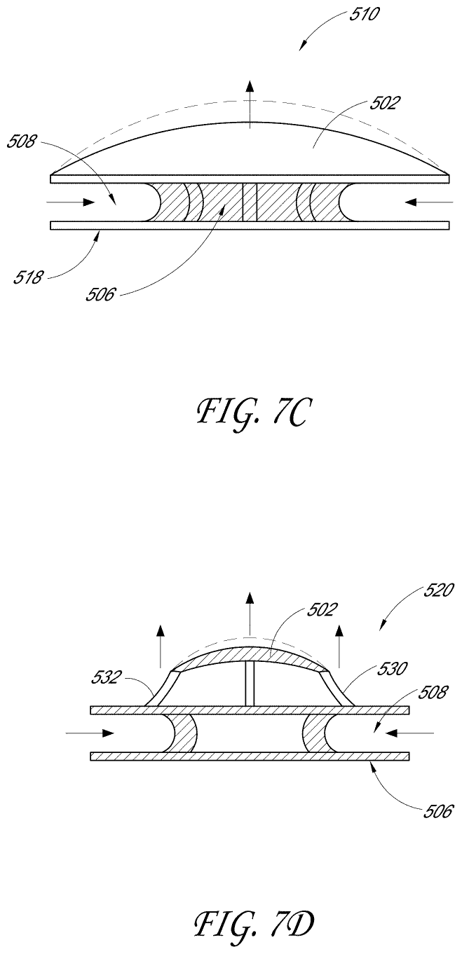

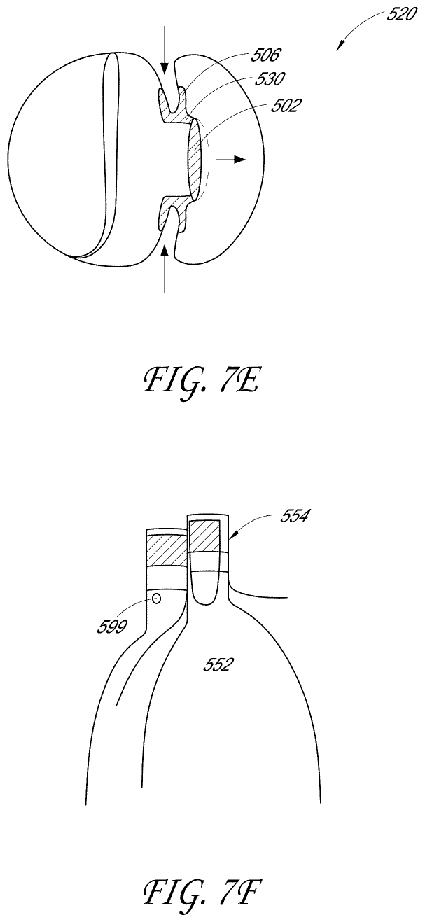

FIG. 7A is a side view of an embodiment of a lens system 500 including a first optic 502 (which can be an anterior optic) and an axially spaced-apart second optic 504 (which can be a posterior optic) connected via a solid 360 degree haptic element 506 in between the first optic 502 and second optic 504. The haptic 506 can include grooves 508 configured to interface with an eye muscle such as the iris, for example. Iris constriction in the direction of arrows can cause anterior displacement of the first optic 502 and reciprocal posterior displacement of the second optic 504 as indicated in phantom, allowing the lens system 500 to provide accommodation.

FIG. 7B is a side view of an embodiment of a dual lens system 509 similar to that of FIG. 7A, with free spaces 511 between interconnecting struts 513 of longitudinally oriented haptic elements 515 rather than a solid haptic element 506 as illustrated in FIG. 7A. The free spaces 511 can advantageously allow for aqueous humor flow therethrough as described elsewhere herein. In some embodiments, the surface area of the free spaces can be about 1%, 2%, 3%, 4%, 5%, 6%, 7%, 8%, 9%, 10%, 15%, 20%, 30%, 40%, 50%, 75%, or more of the surface area of the haptic, or ranges incorporating any two of the aforementioned values.

FIG. 7C is an embodiment of a lens system 510 similar to that of FIG. 7A, except for having only a single optic 502 such as an anterior optic, and the second optic 504 is replaced by a haptic element 518 that forms a posterior portion of grooves 508 configured to interface with an eye muscle such as previously described. FIG. 7D illustrates another embodiment of a lens system 520 with only a single optic 502, a vaulted structure 530 including a plurality of struts 532 connecting the single optic 502 to haptics 506. In the embodiment as illustrated, the walls of the grooves 508 are completely made up of the haptics 506 and spaced axially and posteriorly apart from the single optic 502. FIG. 7E is a cross-sectional side view of an embodiment of a lens system of FIG. 7D. In some embodiments, a vaulted design could include single or a plurality of optics.

FIG. 7F illustrates a lens system 550 including an optic 552 and a haptic 554. The haptic can be configured to slide in a desired direction to allow free movement up to a predetermined threshold spacer 599 distance prior to engaging with an eye muscle such as the iris. The haptic 554 can be positioned along a rail or similar structure in some embodiments and move freely until it faces the limit (e.g., a stop) upon which it will act like a spring based on the material property. The spacing of the rail distance can define when the iris/force engages with the haptic.

In some embodiments, the light reflex-induced changes to pupil diameter may be diminished to improve the effectiveness to side-effect ratio of the lens. Sonic methods will now be described, which could be used alone or in combination. In some embodiments, the lens system could filter out most sensitive wavelengths (e.g., blue light). The lens system could, alternatively or in addition, provide a flat filter for other wavelengths. Natural lenses fortunately increase filtering with age, so the filter is unlikely to be perceived as a loss of vision/brightness for older people. Furthermore, the lens can be configured such that the majority of the IOL optical power change can occur in the small pupil diameter range (e.g. changes between about 2 mm and about 5 mm, or between about 2 mm and about 4 mm, or about 1 mm, 1.5 mm, 2 mm, 2.5 mm, 3 mm, 3.5 mm, 4 mm, 4.5 mm, 5 mm, 5.5 mm, or 6 mm, or ranges including any two of the aforementioned values, which should limit fluctuations that occur with light changes.

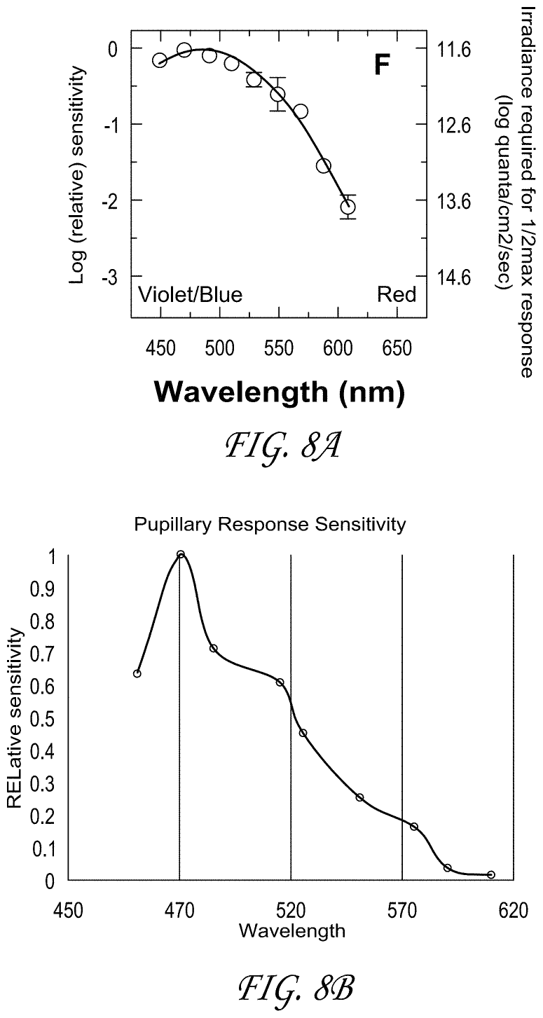

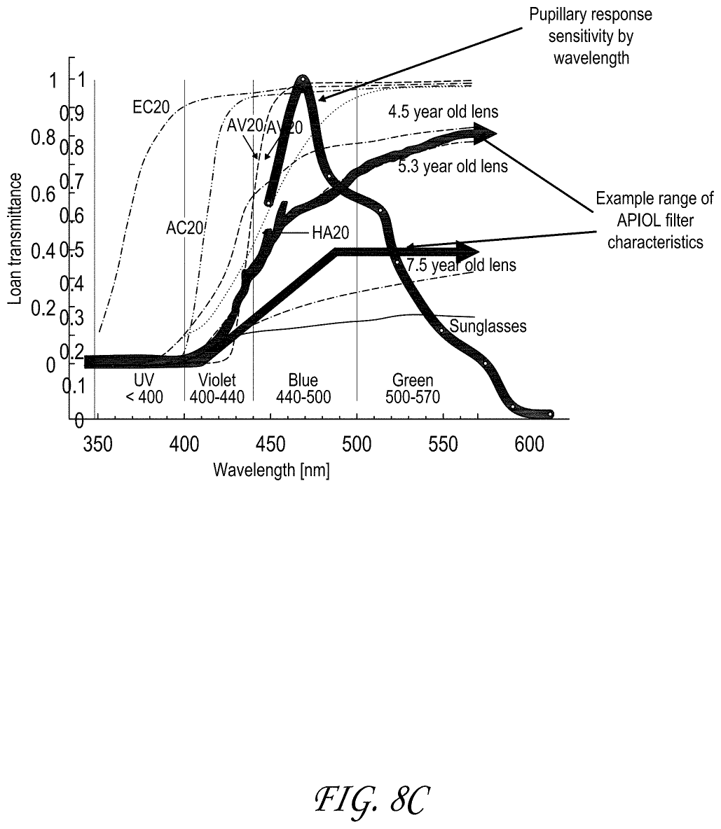

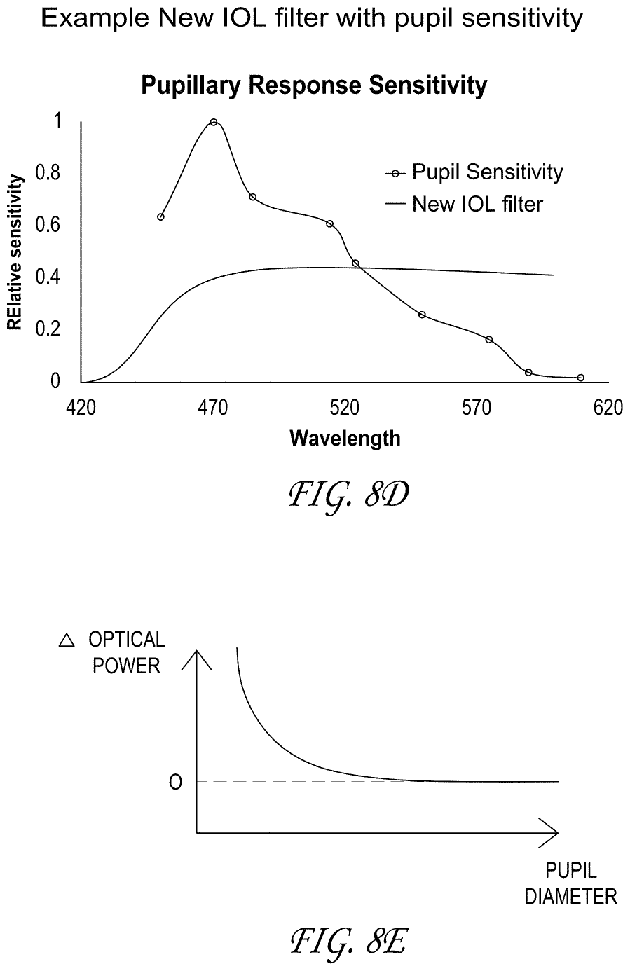

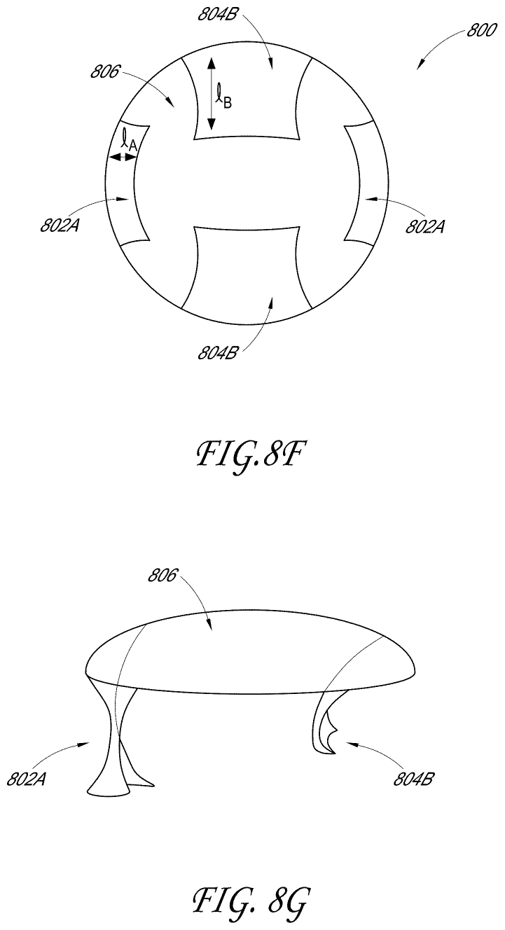

Spectral effects can have important effects on the Pupillary Accommodation Reflex. The pupil can be significantly more sensitive to blue and green light compared with red light. The plot of FIG. 8A shows that the pupil is about 100× more sensitive to blue light than red light—and greatest sensitivity to short wavelength light; and a marked insensitivity to long wavelength light. The rods are the primary driver of pupil diameter in steady state (cones produce transient, but not lasting diameter changes). Melanopsin can also be a significant contributor. FIG. 813 is another graph showing that pupillary response relative sensitivity has a generally linear response curve, and decreases for increasing wavelengths of light.

In some embodiments, a lens system can include a chromatic filter selective to short wavelengths, which can advantageously reduce much of the pupillary light response. 8C illustrates non-limiting example ranges of lens system filter characteristics. lri some embodiments, the chromatic filter can block violet, blue, and/or green light. In some embodiments, the chromatic filter can block light having a wavelength of between about 400nni and about 570 nm, between about 440 nm and about 570 nm, between about 400 nm, and about 500 nm, between about 400 nm, and about 490 nm, or less than about 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, or ranges incorporating any two of the aforementioned values. FIG. 8D is another diagram illustrating the relative sensitivity of the pupillary response with and without a selective chromatic IOL filter, according to some embodiments, showing advantageous significant Hunting of the pupillary light response reflex. The accommodating lens can be customized for a specific patient, including iris factors (e.g., color, pigmentation, thickness, roughness, uniformity, circularity, tissue, age, and other factors unique to the patient), In some embodiments, the specific pupillary response of the patient can be tested, and an appropriate filter can be determined for the lens customized based on the measured pupillary response. Pupil size and range of pupil size in various lighting condition can be an important pre-op biometry to fit an APIOL to the patient. For example, a patient with an upper limit of 5 mm and 2 mm may be fitted with an APIOL with a larger rim/optic diameter than a patient with an upper limit of 4 mm. Iris color or pigmentation can be directly correlated to thickness and compressive force of the iris. Iris tissue with darker pigmentation can be thicker and have more force than tissue with lighter pigmentation. Biometry to examine the patient's iris color/pigmentation, thickness (using ultra-sound imaging) and/or compression force (using a spring gauge implant or deflection implant for example) may be utilized to fit an APIOL.

The lens system can also be configured to have a non-linear response to pupillary diameter. In sonic embodiments, the change in optical power of the lens can be dampened at relatively large pupil diameters to allow for selective movement of the lens to the near-accommodative reflex, as shown in the schematic graph of FIG. 8E. FIG. 8F illustrates a top view of an accommodating lens system 800 with only one optic 806 (or a plurality of optics in other embodiments) and at least a first haptic 802A and a second haptic 804B, the first and second haptics being not identical in one or more features with respect to each other. The differing features could include, for example, one, two, or more of a dimension (e.g., length, width, and/or thickness), material, material property, etc. The different types of haptics within the same lens system can be advantageous in some cases to allow for simultaneous stabilization and accommodation changes at only small pupil diameters (e.g., less than about 5 mm, 4.5 mm, 4 mm, 3.5 mm, 3 mm, or less in some cases). Such configurations can also be desirable to prevent unwanted changes in optical power with fluctuations in pupil diameter at the large diameter end of the scale (e.g., with light intensity changes).

In some embodiments as noted above, haptics 802A maintain centricity over a wide range of pupil diameters, with haptics 804B engaging the eye muscle (e.g., iris) only at smaller diameter pupil sizes to activate the lens power change (e.g., with a shorter and/or relatively softer material). FIG. 8F illustrates two haptics of a first type 802A and two haptics of a second, different type 804B, although other numbers (2, 3, 4, 5, or more of each type; the same or different numbers of each type of haptic) are also possible. In some embodiments, the haptics of each same type 802A, 804B are spaced about 180 degrees apart from each other also other configurations are also possible. In some embodiments the length of the second type of haptics 804B can be greater than the length of the first type of haptics 802A, such as about or at least about 10%, 20%, 30%, 40%, 50%, 75%, or 100% greater in length, or ranges incorporating any two of the aforementioned values. FIG. 8G is a side view of the embodiment of FIG. 8F, with only a single haptic of each type 802A, 804B illustrated for clarity.

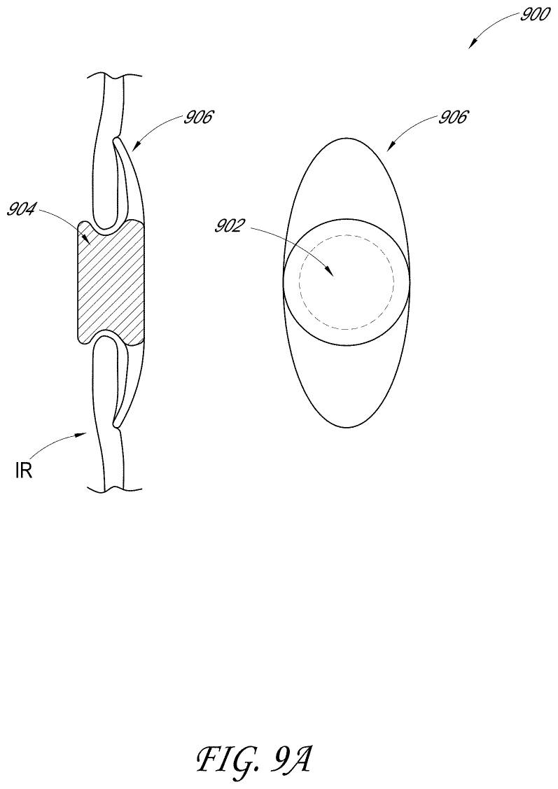

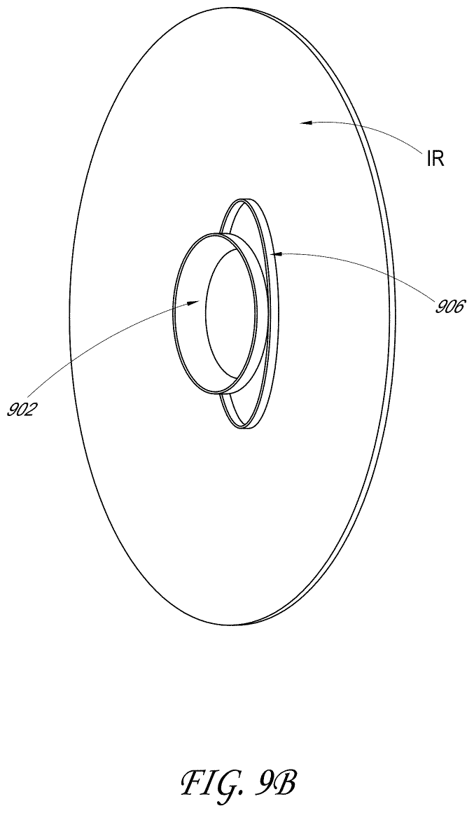

FIGS. 9A-9B illustrate an embodiment of an iris-fixated accommodating pupillary lens, according to some embodiments of the invention. The lens 900 includes at least one optic 902 and a first haptic portion 904 extending posteriorly from the optic and including grooves for connecting with the iris muscle IR. The lens 900 can also include a second haptic portion 906 in the shape of a ring, such as an eccentric ring as shown configured to rest on the anterior surface of the iris muscle IR, FIG. 9B schematically shows an angled top view of the lens in position and resting against the iris IR.

FIGS. 10A-10B illustrate views of another embodiment of an iris-fixated accommodating pupillary lens 1019 including an optic 1000 and a plurality of haptics 1002 extending radially outwardly from the peripheral edge of the optic 1000. There could be 4 haptics 1002 spaced generally 90 degrees apart as illustrated, although any number of haptics 1002 can also be used. The haptics 1002 can include opposing transverse elongate fingers 1004 at their distal ends separated by a space 1006 connected to a more proximal inner channel 1008. The fingers 1004 can be sized and configured to interact with an eye muscle such as the iris IR to anchor the lens 1000 with respect to the iris IR. The lens 1019 can also include larger grooves 1011 in between haptic projections 1002 to allow fluid flow therethrough.

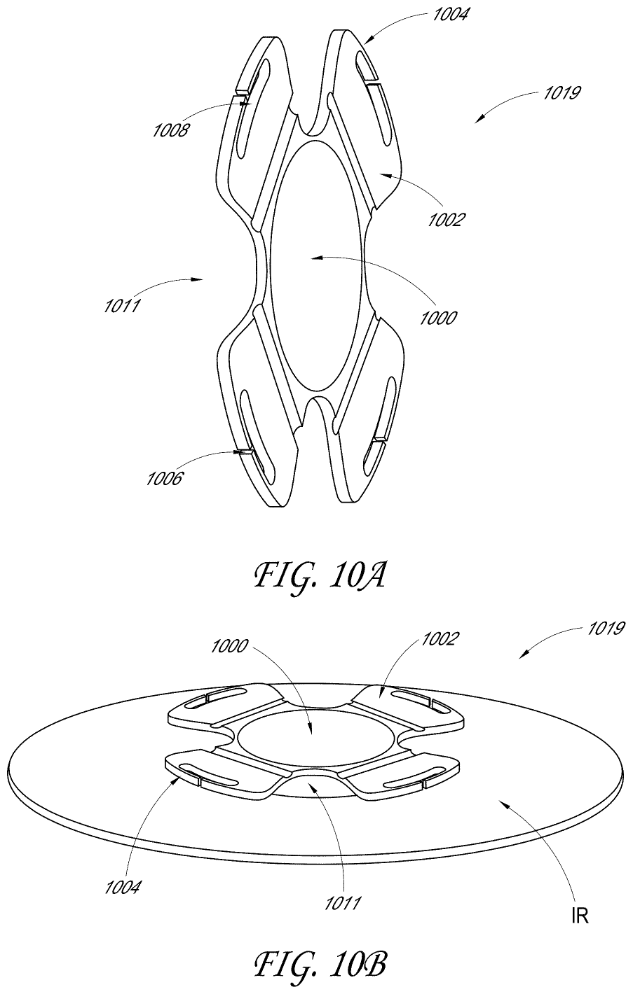

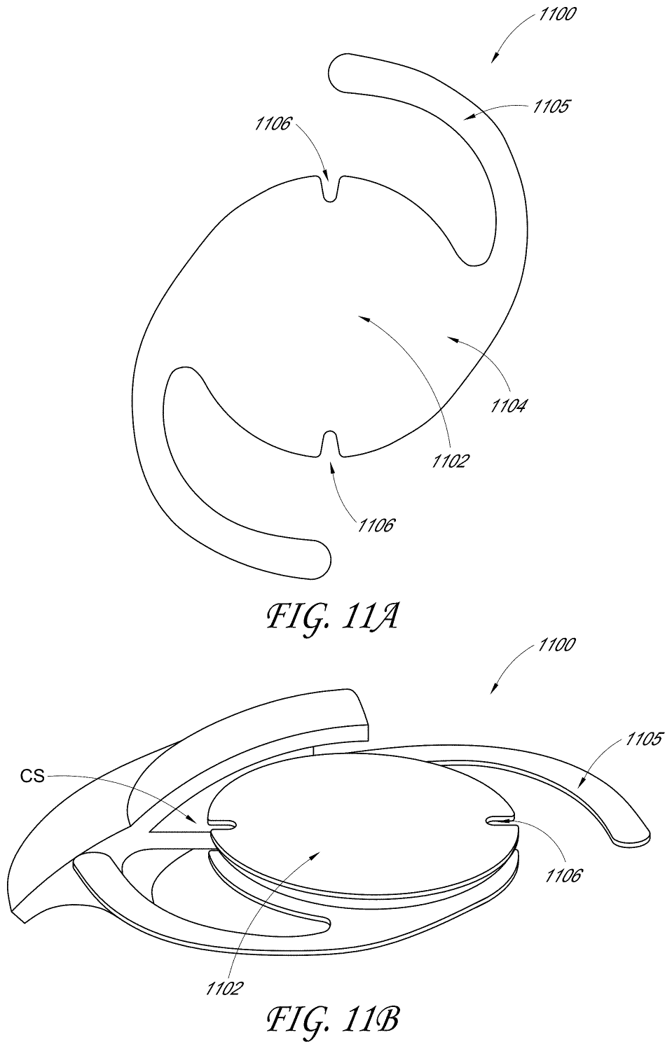

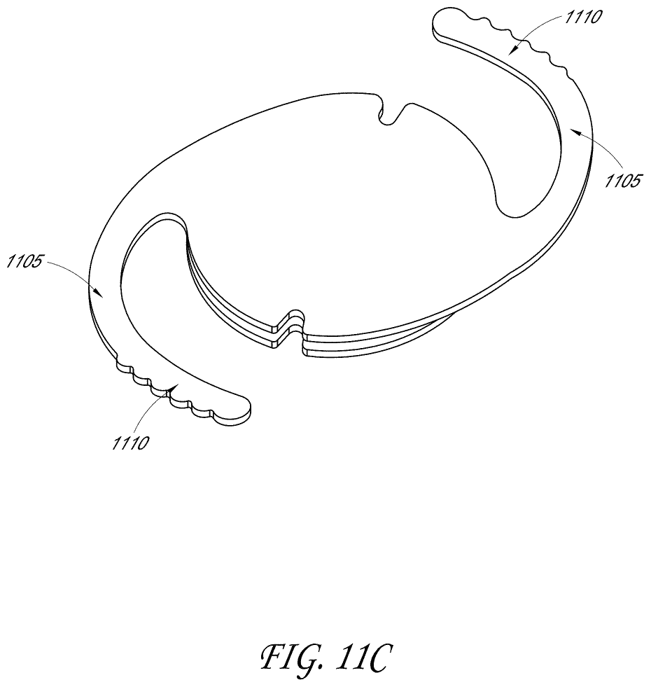

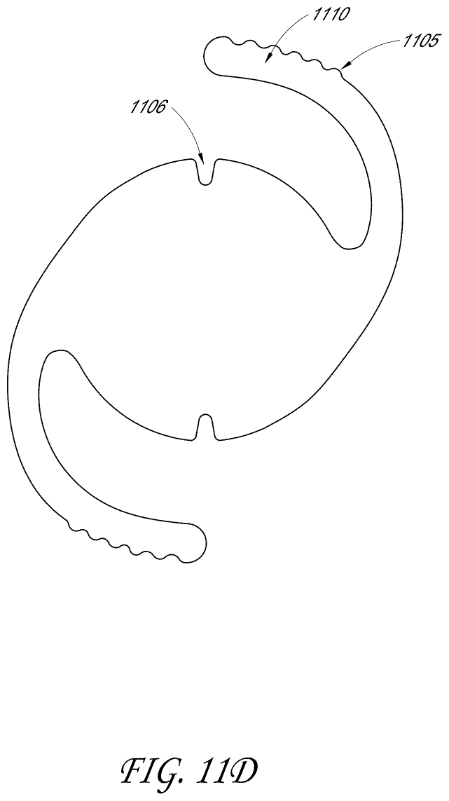

FIGS. 11A-11D illustrate views of an embodiment of an accommodating pupillary lens configured to be placed in the ciliary sulcus. The ciliary sulcus is the space between the posterior surface of the base of the iris and the anterior surface of the ciliary body. As illustrated inr FIG. 11A, the lens 1100 can include one or more optics 1102, and a haptic 1104 with a plurality of lateral grooves, channels, or indentations 1106 to allow aqueous humor flow therethrough. The channels 1106 can be formed in the haptic-optic junction to provide improved flow and lens positioning. The lens could include biconvex surfaces in some embodiments. In some embodiments, the channels can have a dimension, such as a width of between about 400 microns and about 2.5 mm. The channels can also be spread out radially. While two channels are illustrated, 1, 2, 3, 4, 5, 6, 7, 8, 9, 10, or more channels can also be present, or ranges including any two of the aforementioned values. In some embodiments, the optic body diameter and/or rim diameter (where the iris engages) can vary from between about 2 mm and about 7 mm. In some embodiments, the haptic overall length can vary from between about 11 mm to about 15 mm. The haptic thickness can vary in some cases from about 0.5 mm to about 2 mm; lens thickness can vary from about 0.5 mm to about 4 mm; and/or angulation of the haptic can vary from about 0 degrees to about 30 degrees.

The haptic 1104 can include a plurality of radially extending arms 1105 that curve distally along an arc 1108 to assist in anchoring the lens in place. FIG. 11B illustrates the lens 1100 deployed within the ciliary sulcus CS. FIG. 11C illustrates a perspective view and FIG. 11D illustrates a top view of an accommodating pupillary lens, where the radially extending arms 1105 include ridges, undulations, or other attachment features 1110 near the distal end of the arms (e.g., on only one side as shown, or both sides in other embodiments) to better assist in anchoring the lens to the ciliary sulcus CS. The lateral edges of the lens 1100 can in some cases be placed within, e.g., entirely within the ciliary sulcus, and posterior to and not directly attached to the iris. Conventional capsular bag IOU do not require channels as they will not cause pupillary block. In contrast, channels as described herein as well as angulated haptics can be advantageous in some embodiments for placement within the ciliary sulcus. FIG. 11E illustrates a schematic view of a lens system with the haptic 1104 placed in the ciliary sulcus CS for stability and leveraging both the ciliary muscle and the iris. In some embodiments, sulcus placement can promote ease of removal if needed. As illustrated, the haptic 1104 can be spaced apart/positioned away from the posterior iris to avoid chaffing, and provide adequate clearance. In some cases, the haptic 1104 is angulated at about or at least about 5 degrees, 10 degrees, 15 degrees, 20 degrees, or more, or from about 5 degrees to about 15 degrees or more with respect to the iris angle. The posterior optic plane 1198 can be angulated to guide the iris as shown. The anterior optic plane 1196 can also be angulated to guide the iris and minimize optic distortions (e.g., optic diameter much greater than the pupil diameter). The optic 1102 can extend to the pupillary plane, where it allows the iris IR to slide in and out, constricting the lens based on the accommodative reflex demand. The iris IR can make contact with the optic 1102, such as at the rim of the optic. Flow channels 1106 are shown via dotted lines. Table 1 below illustrates non-limiting examples of selected dimensions of elements of a lens system shown schematically in FIG. 11F.

TABLE 1

| Abb. | Diameter | Explanation | Example Range | | Example Nominal | |
|---|---|---|---|---|---|---|
| OD | Optic diameter | Anterior or posterior | 3-7 | mm | 5.5 | mm |
| | | optic diameter | | | | |
| RD | Rim diameter | Rim is the groove or inner | 2.5-6 | mm | 4.5 | mm |
| | | cylinder plane where the | | | | |
| | | iris will make contact | | | | |
| RW | Rim width | Groove thickness where | 0.2 to 1.5 | mm | 0.75 | mm |
| | | iris makes contact | | | | |
| OAL | Overall length | diameter (including | 11-16 | mm | 14 | mm |
| | | haptic end-end) | | | | |
| HT | Haptic thickness | Thickness of the haptic | 0.2 to 1.5 | mm | 0.75 | mm |
| CD | Channel/hole | Channel for aqueous | 0.25 to 2 | mm | 0.75 | mm |
| | diameter | flow to avoid pupillary | | | | |
| | | block | | | | |

FIG. 11G illustrates an embodiment of a solid accommodating pupillary intraocular lens system including an optic and a C-loop haptic configured to be positioned in the haptic similar to that illustrated and described in connection with FIGS. 11A-11D above, Shown are channels 1106 for aqueous flow to avoid pupillary block while still engaging the iris. The rim (e.g., peripheral edge) 1190 separates the anterior and posterior optic plane to capture the iris. FIG. 11H illustrates an embodiment of an accommodating pupillary intraocular lens system similar to that of FIG. 11G, but instead of a solid optic that is not filled with media (e.g., gel or fluid), some embodiments as shown can include an optic 1170 filled with a gel or fluid. Also illustrated are undulations 1110 on the anus 1105 of the haptic to provide stability when deployed in the sulcus CS, as well as flow channels 1106 as previously described.

FIG. 111 illustrates that an accommodating pupillary intraocular lens system can be transformable from a first resting configuration (left side of figure) with a first, larger optic diameter OD to a second configuration (right side of figure, haptic arms not shown for clarity) upon accommodative reflex/iris compression (shown by opposing arrows) on the rim of the optic, which transforms the optic to a second, smaller optic diameter OD′ without affecting or substantially affecting the haptic configuration. The power of the lens increases by reducing the radii of curvature for the optic (e.g., anterior and posterior optic).

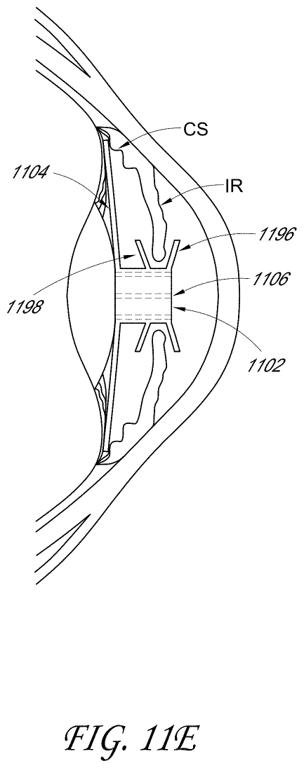

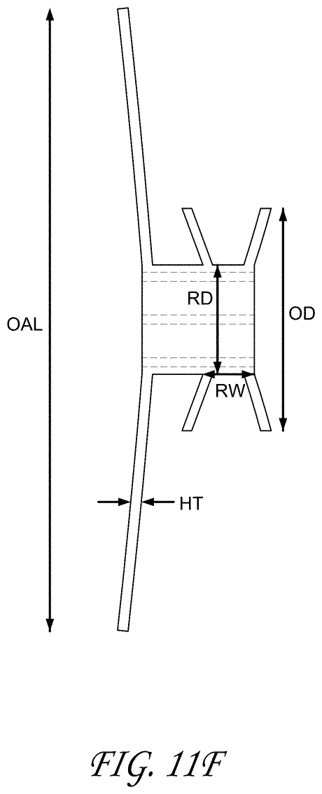

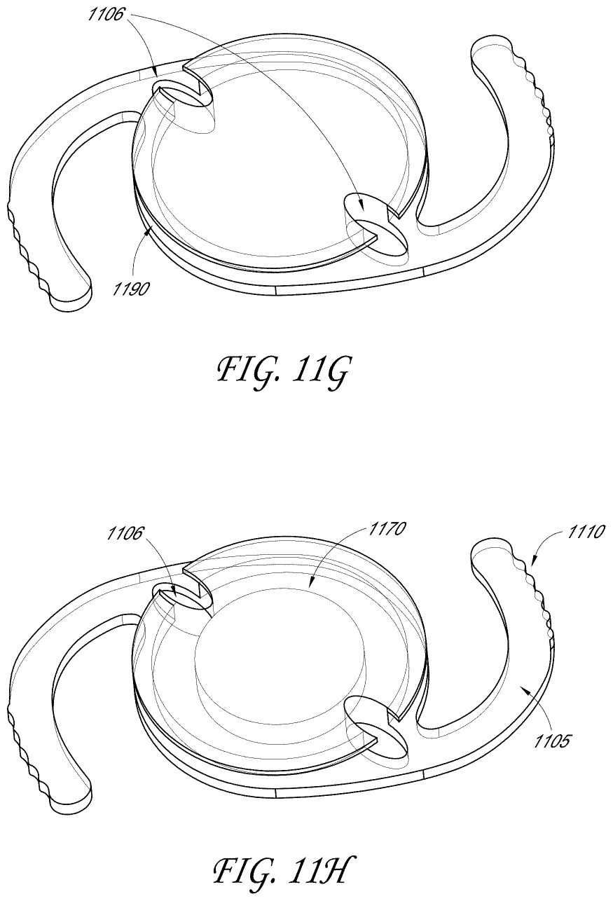

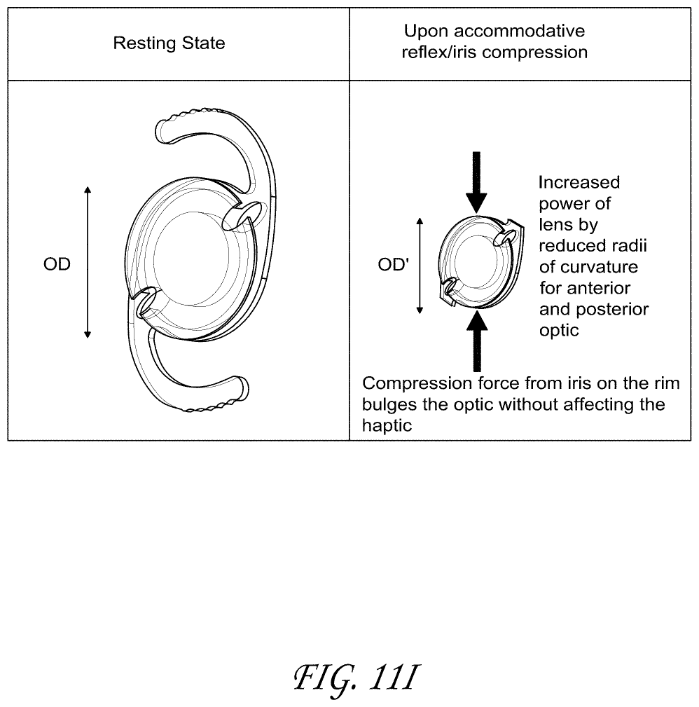

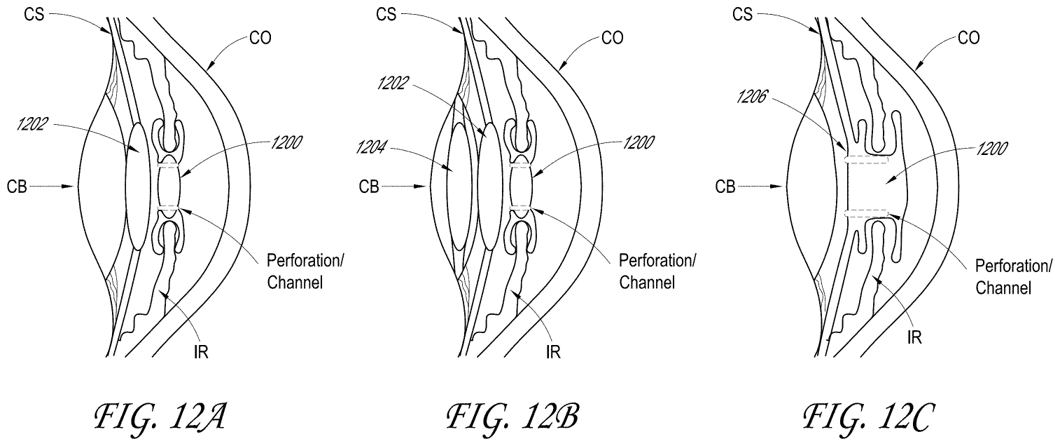

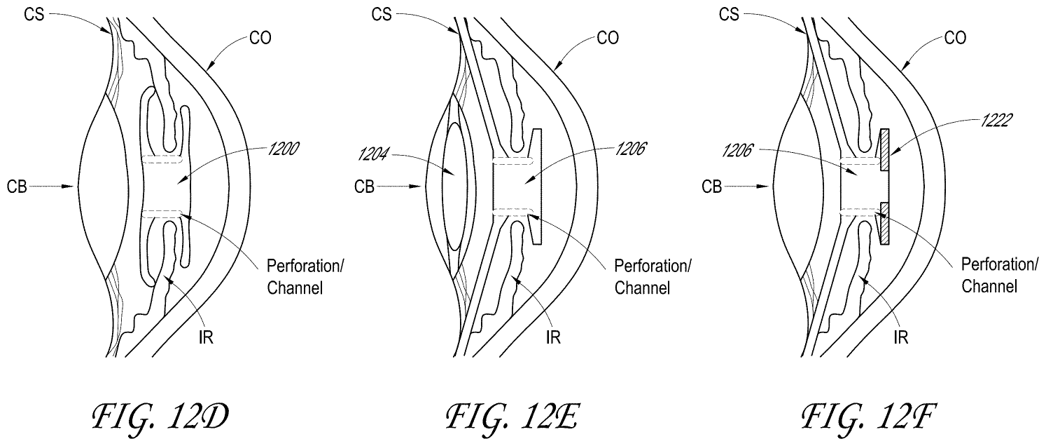

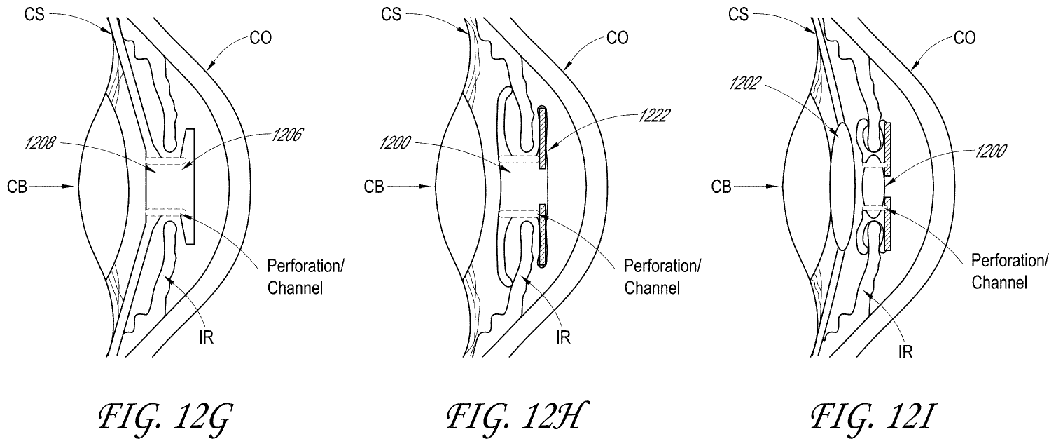

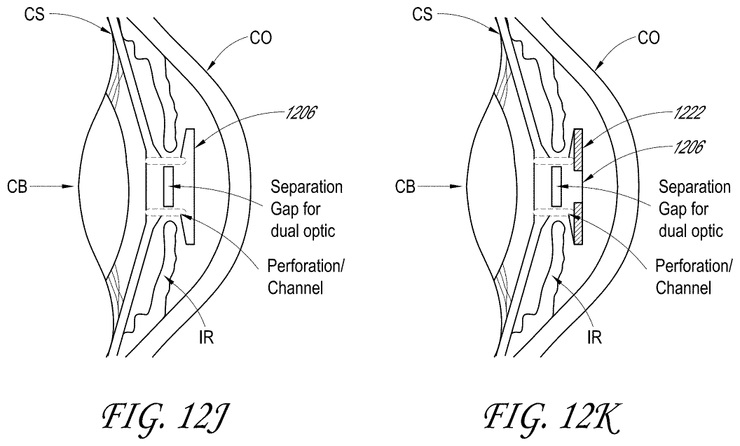

FIGS. 12A-12K illustrate various embodiments of accommodating pupillary lens systems, each showing the cornea CO, iris IR, capsular bag CB, and ciliary sulcus CS as background anatomy. Each lens can have one or more perforations and/or flow channels, that can be axially oriented in some cases and shown in dotted lines on the figures. FIG. 12A illustrates a first lens 1200 attached to the iris IR, and a second lens 1202 attached to the ciliary sulcus. FIG. 12B illustrates a first lens 1200 attached to the iris M., a second lens 1202 attached to the ciliary sulcus CS, and a third lens 1204 (either a natural or artificial IOL) attached within the capsular bag CB. FIG. 12C illustrates a lens 1206 attached to both the ciliary sulcus CS as well as the iris IR. FIG. 12D illustrates a lens 1200 attached to the iris IR only. FIG. 12E illustrates a first lens 1206 attached to both the ciliary sulcus CS as well as the iris IR, and a second lens 1204 (either a natural or artificial IOL) attached within the capsular bag CB. FIG. 12F illustrates a first lens 1206 attached to both the ciliary sulcus CS as well as the iris IR, with the anterior surface having part of the optic zone 1222 partially or completely blocked off to visible light to reduce photophobia while improving accommodation. The lens material may have part of the optic zone partially or completed blocked off by a photochromic or light-sensitive material/chromophore. FIG. 12G illustrates a first lens 1206 attached to both the ciliary sulcus CS as well as the iris IR, and including a central aperture 1208 through the optic and/or the haptic to allow for aqueous humor flow therethrough. FIG. 12H illustrates a first lens 1200 attached to the iris, and an anterior film layer 1222 for light blocking as discussed above. FIG. 12I illustrates an embodiment of a lens similar to FIG. 12H, and also including a second lens 1202 attached to the ciliary sulcus CS. FIG. 12J illustrates a dual optic APIOL system with haptics positioned in the ciliary sulcus CS. There can be a separation gap as shown between the anterior optic and the posterior optic. FIG. 12K is a dual optic APIOL system similar to FIG. 12J, and also including parts of the optic zone 1222 partially or completely blocked off to visible light via, e.g., a film layer to reduce photophobia while improving accommodation as described for example in connection with FIGS. 12F and/or 12H.

In some embodiments, disclosed herein is a lens system for and method of treating presbyopia by placing an accommodating pupillary lens within the eye of a subject in need thereof The lens system can be made of two, three, or more different materials that can provide the change in optical properties including power of the lens by change in shape/vault in response to forces from the iris, ciliary body, or both. Some or all of the materials can be configured to change their properties (e.g., by polymerization) upon delivery of an external energy source or chemical (e.g. light source). The material property change can serve to adjust the optical, refractive, and/or mechanical properties of the lens. The material properties can be changed prior to and/or after implantation. The material property change can serve to adjust for regular or irregular astigmatism.

In some embodiments, the lens system can include an external covering, e.g., a skin layer that partially or fully encloses a volume of deformable media such as a fluid, gel, or other material that is more sensitive to load. The media can be present within the skin prior to implantation, or added within the skin after implantation (e.g., using a needle or cannula).

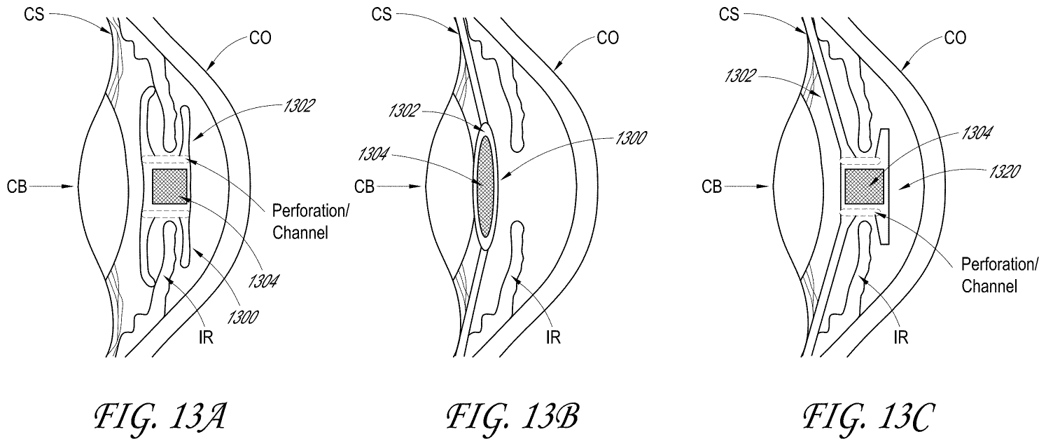

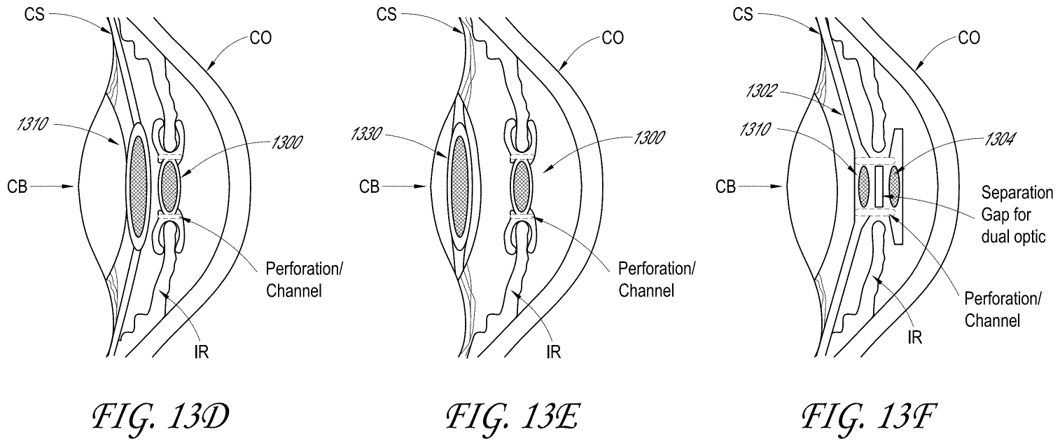

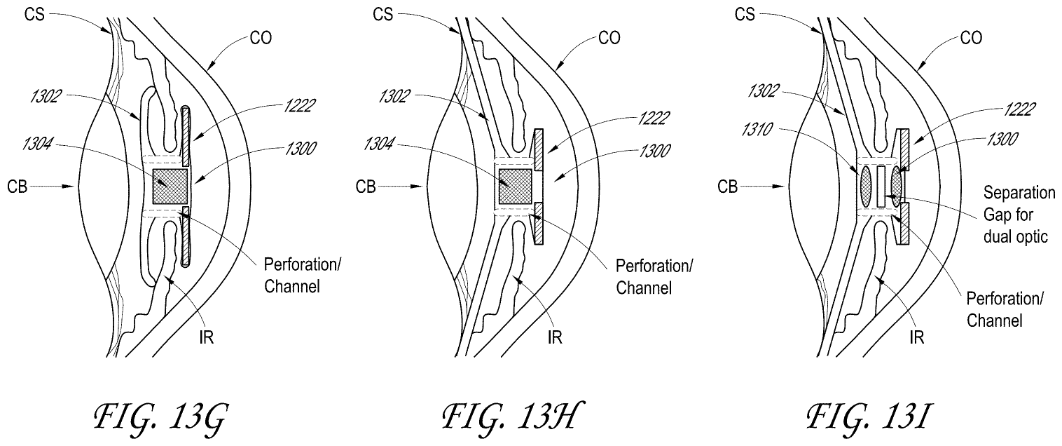

FIGS. 13A-13I illustrate various embodiments of accommodating pupillary lens systems, each showing the cornea CO, iris IR, capsular bag CB, and ciliary sulcus CS as background anatomy. 13A illustrates a lens 1300 including a skin 1302, the lens attached to a portion of the iris IR. The lens, e.g., the optic and haptic elements can be filled with media 1304 such as a fluid, gas, or gel. In some embodiments, the media 1304 can have low viscosity at room temperature and a high refractive index. In some embodiments, the fluid can be a liquid having a viscosity of 1,500, 1,400, 1,300, 1,200, 1,100, 1,000 cP or less at about 23° C. (or between about 100 cP and about 1,200 cP, between about 100 cP and about 1,000 cP, or between about 250 cP and about 1,000 cP in some cases) and a refractive index of at between about 1.3 and about 1.5 in some cases, or about, at least about, or no more than about 1.30, 1.35, 1.40, 1.45, 1.50, or ranges including any of the aforementioned values. The fluid may be a polymer, such as a silicone polymer, such as a phenyl siloxane polymer. In some embodiments where the fluid is made of a polymer, the polymer may or may not be cross-linked and the polymer may be linear or branched.

FIG. 13B illustrates a lens 1310 including a skin 1302 filled with media 1304 and attached to the ciliary sulcus CS. FIG. 13C illustrates a lens 1320 with a skin 1302 filled with a media 1304, the lens 1320 attached to both the iris IR and the ciliary sulcus CS. FIG. 13D illustrates a first lens 1300 attached to the iris IR, and a second lens 1310 attached to the ciliary sulcus CS, either of both lenses of Which can be filled with media 1304 as previously described. FIG. 13E illustrates a first lens 1300 attached to the iris IR and a second lens 1330 attached within the capsular bag CB, either of both of which can be filled with media 1304 as previously described. FIG. 13F illustrates a first lens 1300 and a second lens 1310 with common haptic attachments to both the iris IR and the ciliary sulcus CS. FIG. 13G illustrates a first lens 1300 including a skin 1302 with media 1304 contained within the skin 1302, the first lens 1300 attached to a portion of the iris IR as described in connection with FIG. 13A, and also including an anterior surface having part of the optic zone 1222 partially or completed blocked off to visible light to reduce photophobia while improving accommodation. The lens material may have part of the optic zone partially or completed blocked off by a photochromic or light-sensitive material/chromophore as previously described. FIG. 13H illustrates a lens system similar to that of FIG. 13G except that the lens is positioned/anchored in the ciliary sulcus CS and spaced apart from the posterior iris as described elsewhere herein. FIG. 131 illustrates a lens system similar to FIG. 13H with dual optics 1300, 1310 spaced apart via a separation gap as illustrated.

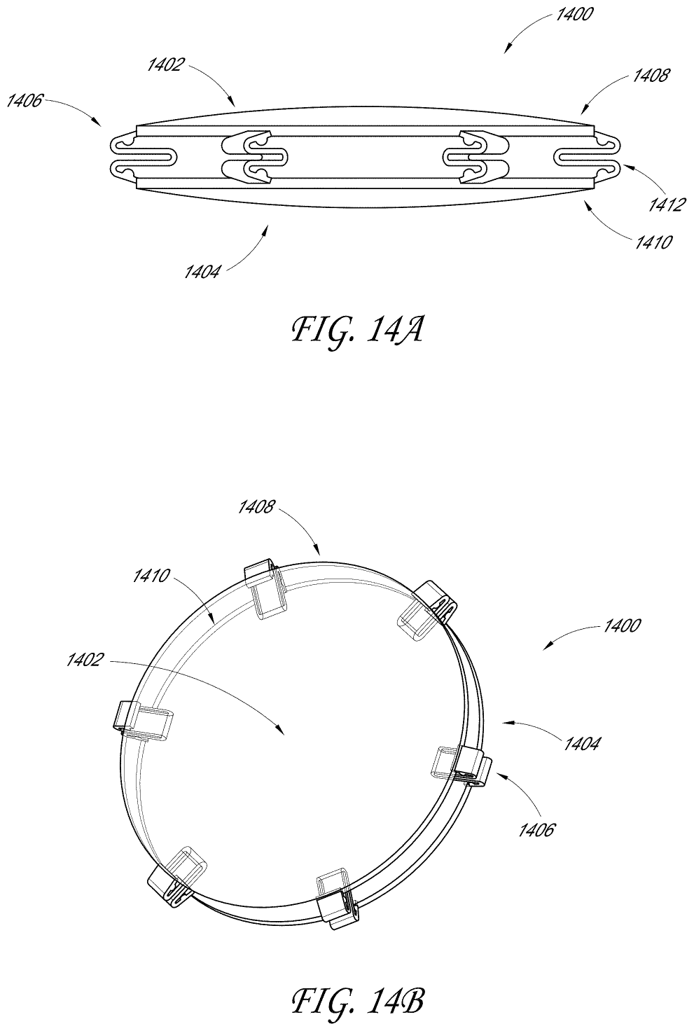

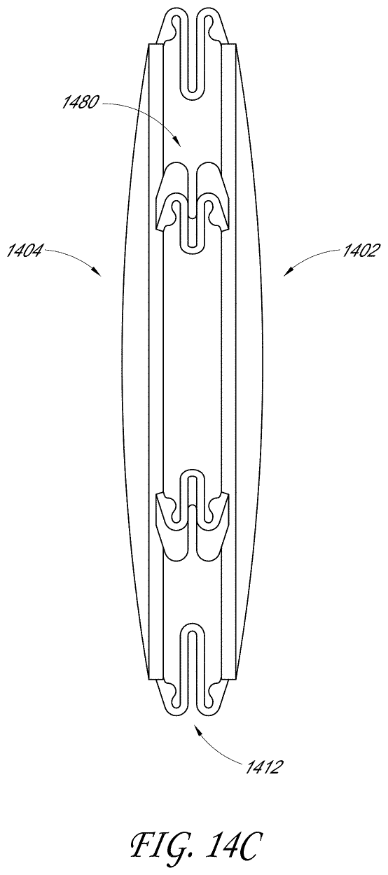

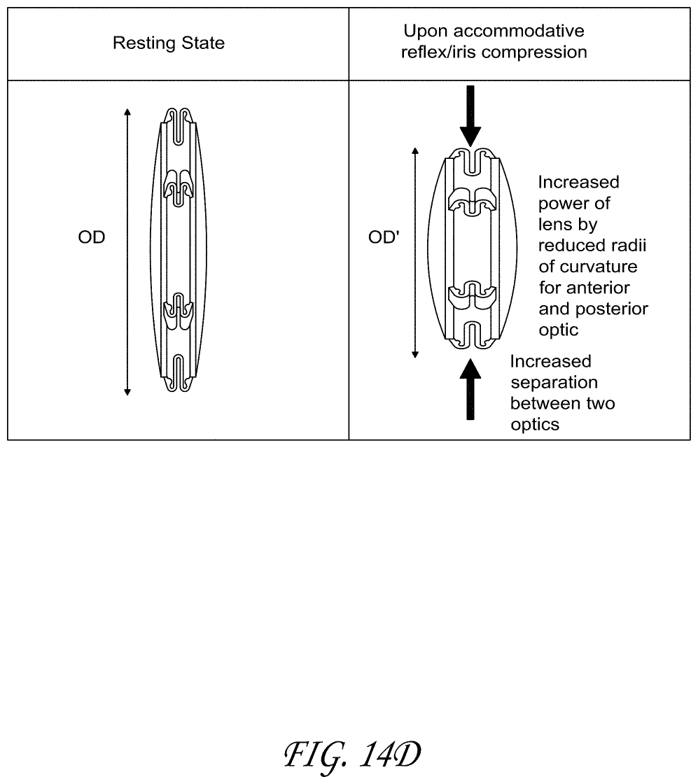

FIGS. 14A-14B illustrate another embodiment of an accommodating pupillary lens 1400 with a dual vault. FIG. 14A is a side view, while FIG. 14B is an angled top view. The system can include biconvex opposing dual optics 1402 and 1404 spaced apart via hinges 1406 (haptic not shown for simplicity, can include haptic configurations including others as described herein). The lens can include a dual ring structure 1408, 1410 and spaced-apart hinges or struts 1412 in an undulating or other pattern. The struts 1412 can be rigid, or flexible and act have a spring force in other embodiments, and can facilitate transformation of the optics 1402, 1404 depending on contraction of the iris and/or ciliary body in some cases, as well as aqueous humor flow through the struts 1412. The struts 1412 can have a loop component that points radially inwardly as shown, and/or moves radially in an appropriate direction, such as inwardly as shown upon compression of the optics 1402, 1404 closer together (or outwardly in other embodiments). The system can include any number of struts, including 6 as shown, or about, at least about, or no more than about 1, 2, 3, 4, 5, 6, 7, 8, 9, 10, or another number of struts 1412 in other embodiments, or ranges including any two of the aforementioned values. The struts 1406 can have hinge-like properties to reduce hoop stress and allow transfer or compression force (from the iris) to transform the optic (1402 and 1404) and separate the distance, thus changing the power of the lens system. Aqueous humor can flow freely between the spacing between the two optics 1402, 1404. The lens system can be positioned as-is in the pupillary margin or with haptics in the sulcus, etc. as described previously. FIG. 14C is another side view illustrating the anterior optic 1402, posterior optic 1404, open space 1480 between the two optics to allow for aqueous free flow as shown schematically via arrows at 1480, and struts/hinges 1412 as previously described and configured to capture the compressive force from the iris to vault/change the curvature of the optics 1402, 1404. FIG. 14D illustrates that an accommodating pupillary intraocular lens system can be transformable from a first resting configuration (left side of figure) with a plurality of optics having a first, larger optic diameter OD to a second configuration (right side of figure, haptic not shown for clarity) upon accommodative reflex/iris compression (shown by opposing arrows) on the struts/hinges between the anterior and posterior optics, which transforms the optics to a second, smaller optic diameter OD′ and increases the length of the hinges and the corresponding separation distance between the two optics. The power of the lens increases by reducing the radii of curvature for the optic (e.g., anterior and posterior optic).

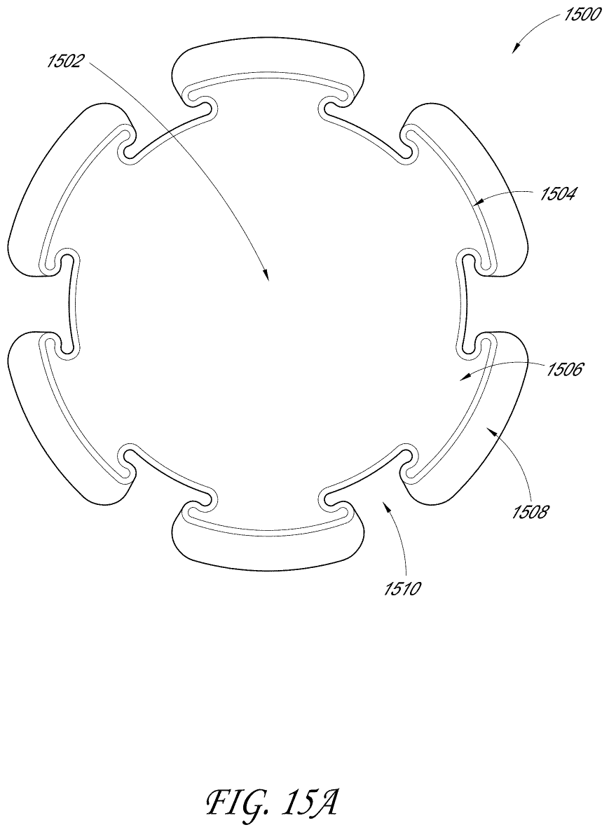

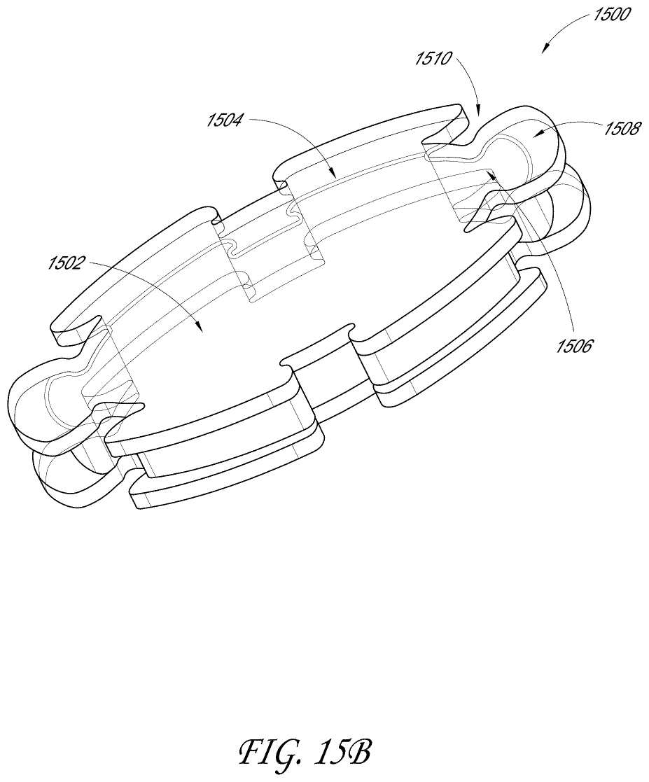

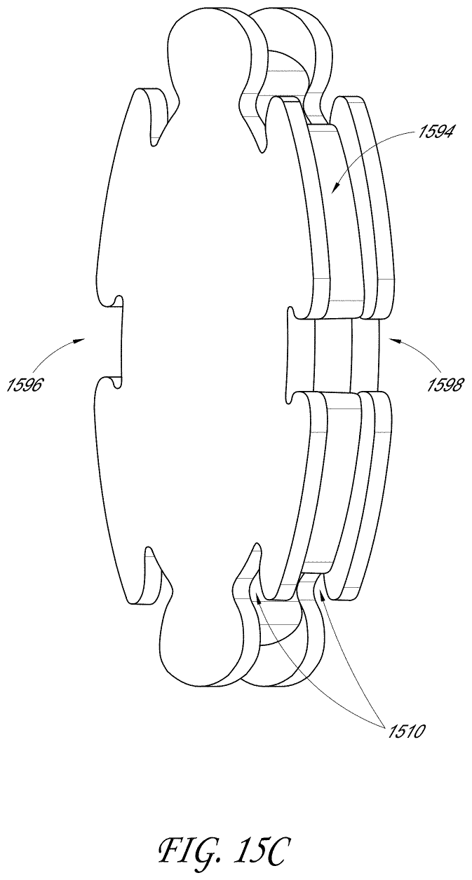

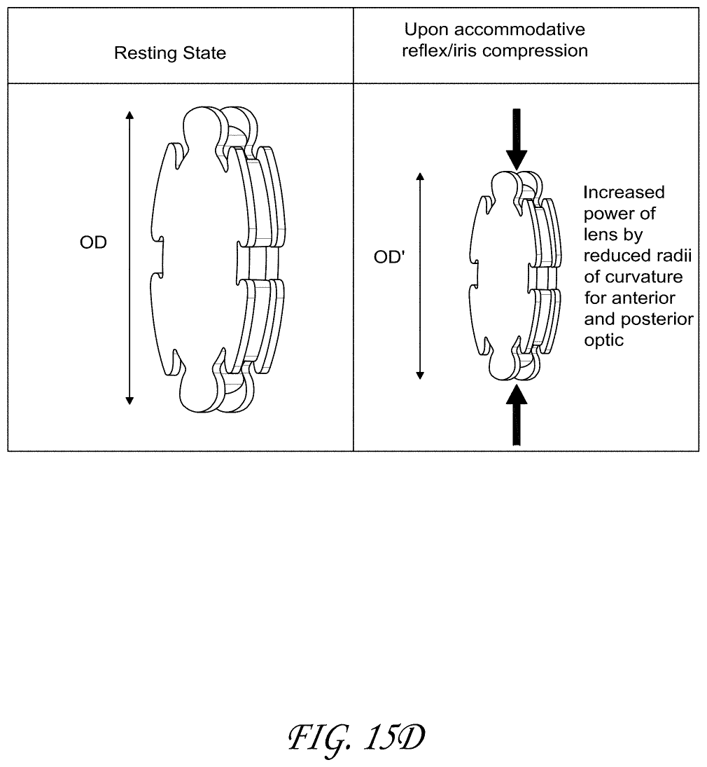

FIGS. 15A-15B illustrate another embodiment of an accommodating pupillary lens 1500 with one or a plurality of optics 1502 and spaced-apart haptics 1504 extending radially outward from the optic 1502. The haptics can include smaller-diameter proximal portions 1506 and larger diameter distal portions 1508 akin to a mushroom or kidney shape defining channels 1510 configured for aqueous flow therethrough and be configured to rest against or otherwise attached to the iris, ciliary body, or other ocular structures. The haptics can take the shape of clover/kidney shaped extrusions. The haptics can in some cases be configured to minimize hoop stress during compression and allow transfer of force/displacement to the optic. The optic can be, for example, one solid or gellfluid-filled design or two separated discs. Additionally, the grooves/space can allow for fluid transfer as well. FIG. 15C illustrates a side perspective view showing the anterior optic plane 1598, posterior optic plane 1596, rim 1.594 where the iris can engage, and channels 1510 in between sections of rim 1594. FIG. 15D illustrates that an accommodating pupillary intraocular lens system can be transformable from a first resting configuration (left side of figure) with a plurality of optics having a first, larger optic diameter OD to a second configuration (right side of figure) upon accommodative reflex/iris compression (shown by opposing arrows) on the rim sections between the anterior and posterior optics, which transforms the optics to a second, smaller optic diameter OD′. The power of the lens increases by reducing the radii of curvature for the optic (e.g., anterior and posterior optic).

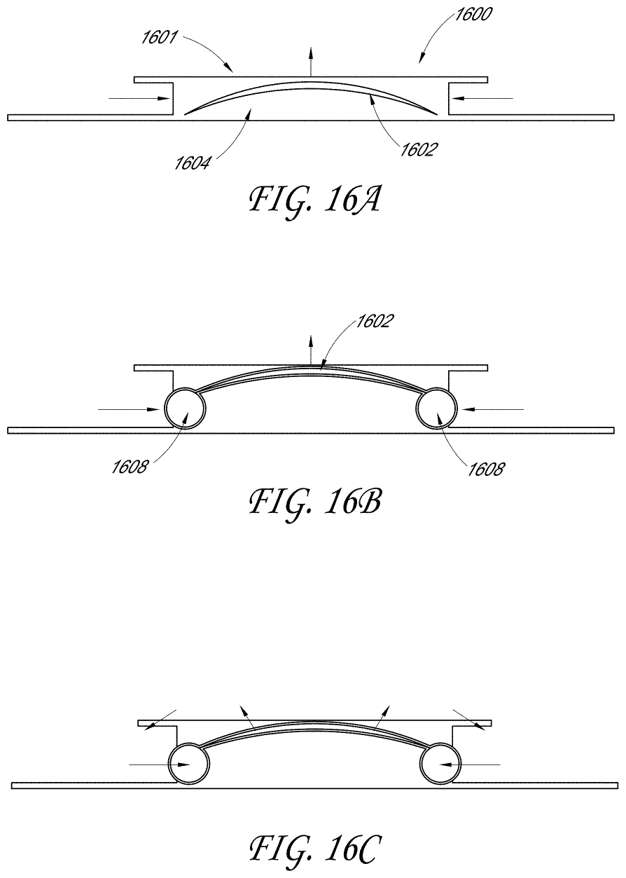

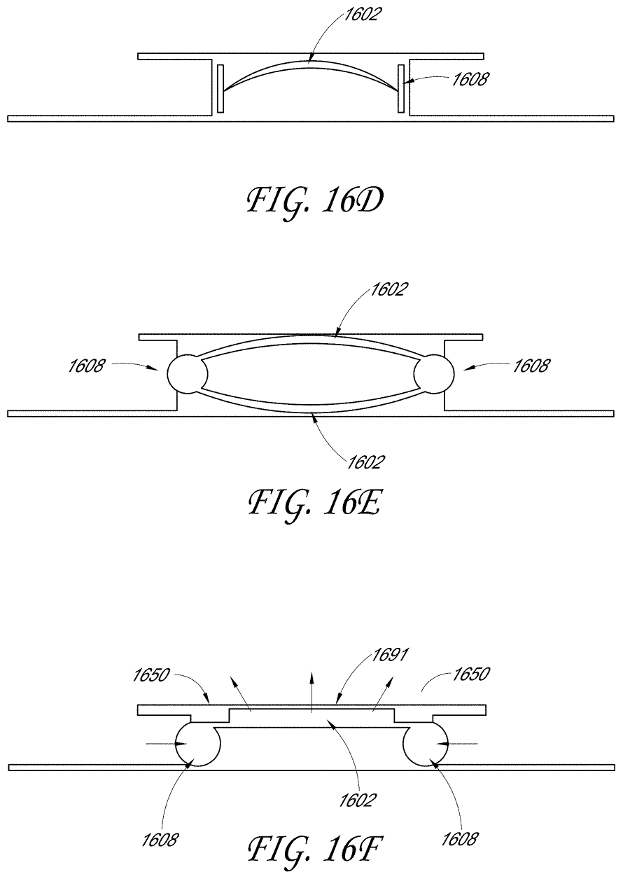

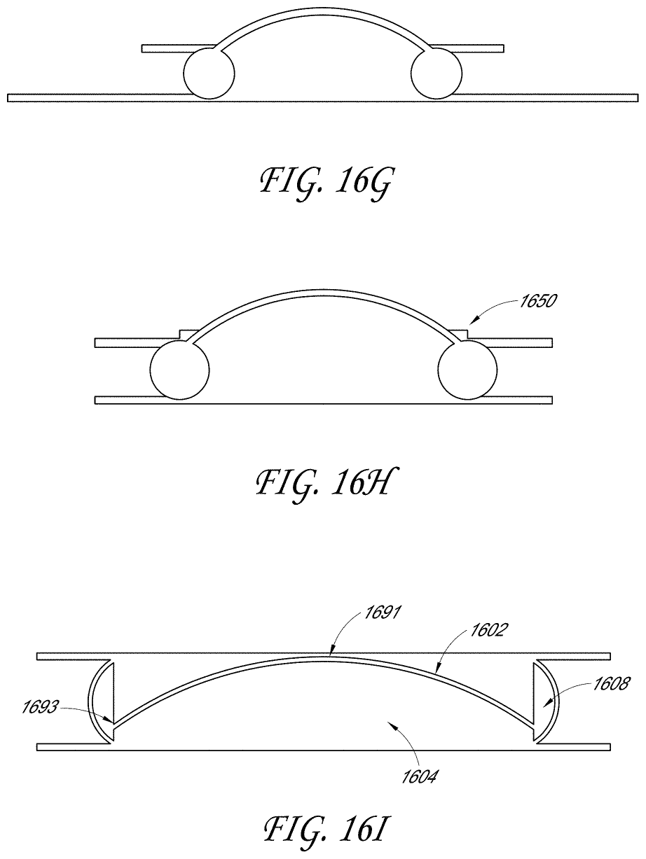

FIGS. 16A-16H illustrate further schematic illustrations of embodiments of an accommodating pupillary lens system. FIG. 16A shows a lens system 1600 with one or a plurality of optics 1601, meniscus portion 1602, which can be an interior zone filled with a gas (e.g., air), gel, liquid (e.g., saline or water), a combination thereof, or other media and surrounded by a bulk material zone 1604, the bulk material different from the media of the meniscus portion 1602. The thin, anterior central region of the meniscus portion 1602 can bias movement of the optic and thus effect curvature change upon compression of the rim by the iris. FIG. 16B illustrates a system 1610 similar to that of FIG. 16A and also including one, two, or more peripheral bladders 1608 which can serve as a reservoir for a volume of the meniscus media. FIG. 16C schematically illustrates via arrows potential movement of the system upon compression (including the thin anterior central region) as previously described. FIG. 16D illustrates a system similar to that of FIG. 1613 with more linear-oriented bladders 1608. FIG. 16E illustrates a system that includes a plurality of anterior and posterior menisci 1602 that can be advantageous for a dual optic system, for example. Each meniscus 1602 can be fluidly connected to bladders 1608. In some embodiments, both menisci 1602 are fluidly connected via the plurality of bladders 1608. In other embodiments, each menisci 1602 has its own separate bladder 1608 and do not fluidly connect with each other. FIG. 16F illustrates a system including a reinforcing ring 1650 that can be configured to force media to the center 1691 of the optic to further affect the curvature change of the optic upon compression. FIG. 16G illustrates that a system that includes a meniscus portion 1602 in a radially compressed configuration, media is forced into the center of the optic, disproportionately affecting the outer radius 1662, curving the optic and changing the power of the optic. In the resting state (not shown) the inner radius 1660 and outer radius 1662 of the meniscus be the same or substantially the same. FIG. 16H illustrates that a reinforcing ring 1650 can further force fluid to the center and control the disproportionate change in the outer radius 1662 upon compression. FIG. 161 illustrates another embodiment illustrating a lens system with peripheral bladders 1608, meniscus portion 1602 surrounded by bulk material zone 1604, with the inner radius 1660 and outer radius 1662 being substantially the same in a resting configuration. In some embodiments, the meniscus zone 1602 can have a progressive or stepwise decrease in thickness from lateral 1693 to the center 1691.

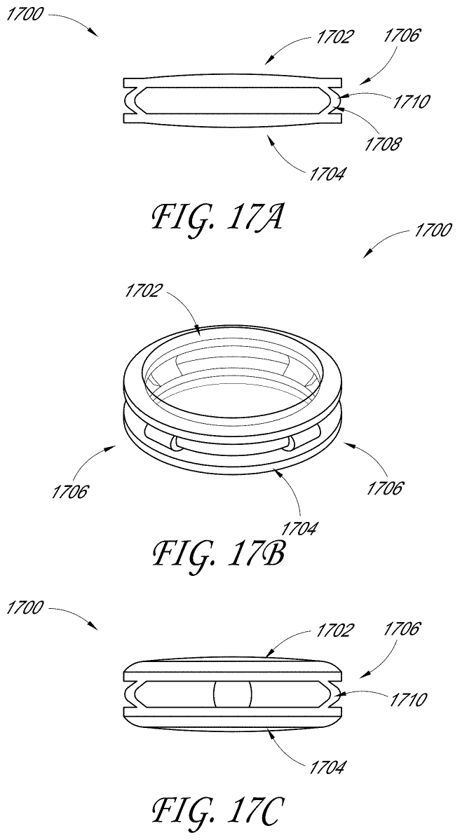

FIGS. 17A-17C illustrate further schematic illustrations of embodiments of an accommodating pupillary lens system 1700 with accordion-like hinges 1706. The system can include a plurality of optics including first optic 1702 and second optic 1704 (which can be anterior and posterior optics respectively) with a plurality of accordion-like hinges 1706 spaced apart from each other and bridging facing surfaces of the optics 1702, 1704. FIGS. 17A-17C illustrate four hinges 1706 generally spaced 90 degrees apart proximate a peripheral edge of each of the optics 1702, 1704, although any number of hinges such as about, at least about, or no more than about 2, 3, 4, 5, 6, 7, 8, 9, 10, or more hinges, or ranges including any two of the aforementioned values can be utilized depending on the desired result. The hinges 1706 can each include a loop section 1708 generally pointing radially outward to an apex 1710 from the center of the system as shown as shown. Forces from the iris, ciliary body, or other structures can bend the hinges in a radially outward direction, and the anterior/posterior surfaces of the optics 1702, 1704 can change radii and move away from each other, increasing power of the lens. In other embodiments, the hinges can be configured to move radially inward upon application of a force rather than radially outward.

In some embodiments, the pupillary accommodating intraocular lens can be placed only in one eye (e.g., monocularly, such that there are no APLs in both eyes) to provide desired balance between 3D binocular vision and distance/near accommodation (e.g., better depth of focus). In some embodiments, monocular placement of a pupillary accommodating intraocular lens can take place in the same, prior to, or after a cataract surgery procedure of which another artificial intraocular lens is inserted into the capsular bag. In some embodiments, the APL can have a power of about or less than about 10, 9, 8, 7, 6, 5, 4, 3, 2, or 1 diopter or ranges including any of the aforementioned values to provide an improved depth of focus. In other embodiments, APLs as disclosed herein can be placed binocularly. In some embodiments, the intracapsular lens placed could be a monofocal, multifocal, or toric IOL.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “inserting an accommodating intraocular lens proximate the peripheral edge of the pupil of the eye” includes “instructing the inserting an accommodating intraocular lens proximate the peripheral edge of the pupil of the eye.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A pupillary accommodating intraocular lens, comprising:
at least one optic configured to refract light and change optical power in response to a change in pupillary diameter when positioned within an eye, thereby facilitating accommodation; and
at least one haptic operably connected to the optic;
wherein the lens is configured to change optical power to a first, greater extent at lower pupillary diameters and to a second, lesser extent at higher pupillary diameters,
wherein the intraocular lens is configured to allow for aqueous humor flow therethrough when the lens is positioned within the eye.

2. The pupillary accommodating intraocular lens of claim 1, comprising a plurality of lateral channels at a haptic-optic junction of the lens to allow aqueous humor flow therethrough.

3. The pupillary accommodating intraocular lens of claim 1, comprising a central aperture in the optic configured to allow aqueous humor flow therethrough.

4. The pupillary accommodating intraocular lens of claim 1, comprising a plurality of perforations in the haptic to allow aqueous humor flow therethrough.

5. The pupillary accommodating intraocular lens of claim 1, wherein the haptic comprises a plurality of spaced-apart elongate members extending radially outwardly from the optic.

6. The pupillary accommodating intraocular lens of claim 1, wherein the haptic comprises at least 4 elongate members extending radially outwardly from the optic.

7. The pupillary accommodating intraocular lens of claim 5, wherein the plurality of elongate members comprise anchoring features on at least one radially outward facing surface.

8. The pupillary accommodating intraocular lens of claim 7, wherein the anchoring features comprise ridges or grooves.

9. The pupillary accommodating intraocular lens of claim 5, wherein the plurality of elongate members comprise curved distal ends.

10. The pupillary accommodating intraocular lens of claim 1, wherein the lens comprises a light filter configured to filter out blue light.

11. The pupillary accommodating intraocular lens of claim 1, wherein the lens comprises a light filter configured to filter out light having a wavelength of between about 400 nm and about 500 nm.

12. The pupillary accommodating intraocular lens of claim 1, wherein the lens is configured to he placed proximate an iris.

13. The pupillary accommodating intraocular lens of claim 1, wherein the lens is configured to be placed proximate a ciliary body or within the ciliary sulcus.

14. The pupillary accommodating intraocular lens of claim 1, wherein the lens comprises a covering, and the covering encloses a volume of media.

15. The pupillary accommodating intraocular lens of claim 14, wherein the media comprises a gel.

16. The pupillary accommodating intraocular lens of claim 14, wherein the media comprises a fluid.

17. The pupillary accommodating intraocular lens of claim 14, wherein the media is polymerizable in situ.

18. The pupillary accommodating intraocular lens of claim 1, wherein the haptic comprises a plurality of elongate members extending radially outwardly from the optic.

19. The pupillary accommodating intraocular lens of claim 1, wherein the optic comprises a meniscus zone surrounded by a bulk material zone.

20. The pupillary accommodating intraocular lens of claim 19, wherein the meniscus zone is filled with a media different from the bulk material zone.

21. The pupillary accommodating intraocular lens of claim 19, wherein the meniscus zone has a central thickness less than that of a lateral thickness.

22. The pupillary accommodating intraocular lens of claim 19, further comprising a reservoir of media fluidly connected to the meniscus.

23. The pupillary accommodating intraocular lens of claim 1, wherein the haptic comprises a plurality of elongate members extending radially outwardly from the optic.

24. A pupillary accommodating intraocular lens, comprising:
at least one optic configured to refract light and change optical power in response to a change in pupillary diameter, thereby thcilitating accommodation; and
a first plurality of haptics comprising the same length and operably connected to the optic; and
a second plurality of comprising the same length and operably connected to the optic;
wherein the intraocular lens is configured to allow for aqueous humor flow therethrough when the lens is positioned within an eye.
wherein the length of the first plurality of haptics is less than the length of the second plurality of haptics,
wherein when implanted the first plurality of haptics continuously contacts the peripheral edge of a pupil while the second plurality of haptics only intermittently contacts the peripheral edge of the pupil at smaller pupil diameters, thereby increasing a change in lens power at smaller pupil diameters.

25. A pupillary accommodating intraocular lens, comprising:
a first optic and a second optic configured to refract light and change optical power in response to a change in pupillary diameter, thereby facilitating accommodation;
a plurality of spaced apart hinges operably connecting the first optic and second optic;
at least one haptic;
wherein the intraocular lens is configured to allow for aqueous humor flow therethrough in a channel between the first optic and the second optic when the lens is positioned within an eye.
wherein radial compression on the hinges causes the first optic and the second optic to move axially apart, thereby changing the power of the optics.

26. A pupillary accommodating intraocular lens, comprising:
a first optic and a second optic configured to refract light and change optical power in response to a change in pupillary diameter, thereby facilitating accommodation;
a plurality of spaced apart projections extending radially outward from the first optic and the second optic, a rim connecting portions of the projections;
wherein the intraocular lens is configured to allow for aqueous humor flow therethrough in channels between portions of the rim when the lens is positioned within an eye,
wherein radial compression on the rim causes the first optic and the second optic to move axially with respect to each other, thereby changing the power of the optics.

27. A method for treating presbyopia or astigmatism in a patient, comprising:
implanting a pupillary accommodating intraocular lens into the eye, the lens comprising at least one optic operably connected to at least one haptic;
positioning the lens such that at least part of a peripheral edge of the optic is proximate the iris and the haptic is positioned in the ciliary sulcus and spaced apart from a posterior portion of the iris such that a change in pupillary diameter results in accommodation of the lens; and
controlling the optical power of the lens in response to a change in pupillary diameter such that the lens changes optical power to a first, greater extent at lower pupillary diameters and to a second, lesser extent at higher pupillary diameters.

28. The method of claim 27, comprising positioning a first lens proximate the iris and a second lens proximate the ciliary sulcus, the second lens posterior to the first lens.

29. The method of claim 27, wherein the lens is non-penetratingly attached to the iris.

30. The method of claim 27, wherein the lens is penetratingly attached to the iris.

31. The method of claim 27, wherein the patient also has an intracapsularens present.

32. The method of claim 27, wherein the patient does not have an intracapsular lens present.

33. The method of claim 27, wherein the lower pupillary diameter is less than about 5 mm.

34. The method of claim 27, wherein the lower pupillary diameter is less than about 4 mm.

35. The method of claim 27, wherein controlling the optical power comprises filtering light of a selected range of wavelengths through a chromatic filter associated with the lens, thereby reducing the pupillary light response.

36. The method of claim 27, wherein controlling the optical power comprises filtering light of a selected range of wavelengths through a chromatic filter associated with the lens, thereby reducing the pupillary light response.

37. The method of claim 27, where the lens comprises a first plurality of haptics and a second plurality of haptics, wherein the length of the first plurality of haptics is less than the length of the second plurality of haptics. wherein after implantation the first plurality of haptics continuously contacts the peripheral edge of a pupil while the second plurality of haptics only intermittently contacts the peripheral edge of the pupil at smaller pupil diameters, thereby increasing a change in lens power at smaller pupil diameters.

38. The method of claim 27, wherein the pupillary accommodating intraocular lens is placed only monocularly, and not binocularly.

39. The method of claim 38, performed in the same operative procedure as positioning another artificial intraocular lens in a capsular bag of the same eye of the patient of which the pupillary accommodating intraocular lens is placed.

40. A method for treating presbyopia or astigmatism in a patient, comprising:
providing a pupillary accommodating intraocular lens, the lens comprising at least one optic operably connected to at least one haptic; and
implanting the lens such that at least part of a peripheral edge of the optic is proximate the iris and the haptic is positioned in the ciliary sulcus and spaced apart from a posterior portion of the iris such that a change in pupillary diameter results in accommodation of the lens.

41. The method of claim 40, further comprising controlling the optical power of the lens in response to a change in pupillary diameter such that the lens changes optical power to a first, greater extent at lower pupillary diameters and to a second, lesser extent at higher pupillary diameters.

42. The method of claim 40, further comprising controlling the optical power of the lens in response to a change in pupillary diameter such that the lens changes optical power to a first, greater extent at lower pupillary diameters and to a second, lesser extent at higher pupillary diameters.
